(19) United States
(12) Patent Application Publication
(10) Pub. No.: US 2016/0220557 A1
(43) Pub. Date: Aug. 4, 2016

(54) USE OF ACETYL-COA CARBOXYLASE INHIBITORS FOR TREATING ACNE VULGARIS

(71) Applicant: PFIZER INC., New York, NY (US)

(72) Inventors: William Paul Esler, Clinton, CT (US); Gabriele Elisabeth Sonnenberg, Cambridge, MA (US)

(73) Assignee: Pfizer Inc., New York, NY (US)

(21) Appl. No.: 15/021,010

(22) PCT Filed: Aug. 29, 2014
(86) PCT No.: PCT/IB2014/064151

§ 371 Date: Mar. 10, 2016

Related U.S. Application Data
(60) Provisional application No. 61/877,058, filed on Sep. 12, 2013.

(51) Int. Cl. A61K 31/4709

(52) U.S. Cl. CPC A61K 31/4709

(57) ABSTRACT

The present invention relates to methods of treating and/or preventing acne in patients comprising the step of administering to patients in need of such treatment a therapeutically effective amount of an ACC inhibitor or a pharmaceutically acceptable salt thereof.

### FIELD OF THE INVENTION

The present invention relates to methods of treating and/or preventing the progression of acne vulgaris (acne) using an acetyl-CoA carboxylase (ACC) inhibitor or pharmaceutical compositions containing such an inhibitor.

### BACKGROUND

Acne vulgaris consists of a spectrum of skin lesions including comedones, inflammatory papules, pustules, nodules and cysts. The disease is classified as mild, moderate or severe depending on lesion severity and anatomical lesion distribution. Disease onset typically occurs at puberty because of elevated sebum production triggered by increased androgen levels. Approximately 90% of adolescents are affected by acne with 15% seeking medical treatment; moreover, the disease continues to be prevalent in 23-35% of young adults (18-28 years). Biologically, acne is considered an inflammatory disease of the pilosebaceous duct with several distinguishing characteristics, including: (a) excess sebum production; (b) abnormal keratinocyte proliferation and desquamation leading to ductal obstruction; (c) proliferation of Propionibacterium acnes (P. acnes); and (d) inflammation. These factors are often interdependent. For example, elevated androgen levels lead to epithelial desquamation and follicular obstruction as well as excess sebum production causing the obstructed follicles to fill with lipid forming comedones. This excess sebum then serves as a substrate for P. acnes bacteria which metabolize the sebum to release free fatty acids that promotes further bacterial replication and inflammation. While multiple factors contribute to the etiology of the disorder, acne cannot occur without sebum as sebum serves as the nutrient source for P. acnes (Smith and Thiboutot, 2008).

Current standard of care for acne includes topical therapies for mild to moderate disease, and systemic therapy for moderate to severe disease. These current therapies are either marginally effective or lack suitable safety profiles for widespread use. Topical acne treatments include retinoids, topical antibiotics, benzoyl peroxides and combinations thereof. Systemic treatments include hormonal therapies, oral antibiotics and isotretinoin (Accutane) (Dawson et al.). Hormonal therapies, including oral contraceptives and androgen receptor blockers, are used in female patients for the treatment of moderate to severe acne with modest efficacy. Oral antibiotics including doxycycline, minocycline, tetracycline and erythromycin are also modestly effective in treating acne, particularly when matched against patterns of P. acnes resistance; although, photosensitivity and gastrointestinal disturbance limit their use (Gannon 2011). Isotretinoin presents a number of serious adverse effects. The agent has a Pregnancy Category X teratogenicity warning, and requires special prescribing precautions and routine pregnancy testing. Additionally, isotretinoin causes severe mucocutaneous toleration issues (dry skin, eyes, nasal passages, lips, etc) which can be dose limiting if not adequately managed with palliative care. Isotretinoin treatment is associated with adverse plasma lipid changes (increased TG, LDL) and hepatic toxicity (ALT/AST elevation requiring liver function testing prior to treatment. Additionally, isotretinoin therapy has also been associated with myalgia (50% of patients have elevated CK levels), calcification of ligaments and detrimental ocular effects (loss of night vision, loss of color vision and eye dryness). In isolated cases, isotretinoin has been associated with neurological/psychological adverse effects including depression, psychosis and potentially suicide.

Therefore, a need exists for a novel approach to treating acne with a favorable efficacy/safety profile. The present invention provides a new therapeutic approach for treating acne comprising the use of ACC inhibitors.

### SUMMARY OF THE INVENTION

The present invention relates to methods of treating and/or preventing acne in patients comprising the step of administering to patients in need of such treatment a therapeutically effective amount of an ACC inhibitor or a pharmaceutically acceptable salt thereof.

In another embodiment, the present invention relates to methods of treating and/or preventing acne in patients comprising the step of administering orally to patients in need of such treatment a therapeutically effective amount of an ACC inhibitor or a pharmaceutically acceptable salt thereof.

In another embodiment, the present invention relates to methods of treating and/or preventing acne in patients comprising the step of administering topically to patients in need of such treatment a therapeutically effective amount of an ACC inhibitor or a pharmaceutically acceptable salt thereof.

In another embodiment, the present invention relates to methods of treating and/or preventing acne in patients comprising the step of administering to patients in need of such treatment a pharmaceutical composition comprising a therapeutically effective amount of an ACC inhibitor, or a pharmaceutically acceptable salt thereof, and at least one pharmaceutically acceptable carrier.

In another embodiment, the present invention relates to methods of reducing sebum triglycerides, sebum free fatty acids, cholesterol esters and sebum waxy esters in patients comprising the step of administering to patients in need of such treatment a therapeutically effective amount of an ACC inhibitor or a pharmaceutically acceptable salt thereof.

### BRIEF DESCRIPTION OF THE DRAWINGS

FIG. 1 shows the percent contribution of de novo synthesized palmitate over time to the palmitate in sebum lipids and to circulating lipids (VLDL triglyceride) in healthy human subjects.

FIG. 2 shows inhibition of de novo lipogenesis by Example 1 (triangles) and Example 3 (circles) relative to vehicle in the human SZ95 sebocyte cell line.

FIG. 3 shows inhibition of ¹⁴C-acetate incorporation into sebum lipids by Example 3 vs. vehicle in the human SZ95 sebocyte cell line. The sebum lipid species were separated by thin layer chromatography and visualized using autoradiography.

FIG. 4 shows sebum production in healthy human volunteers treated with Example 1 (200 mg BID) or placebo for 14 days as assessed using Sebumeter®. Data are expressed relative to baseline measures.

FIG. 5 shows the change in triacylglycerol, wax esters and free fatty acids in healthy human volunteers treated with Example 1 (200 mg BID) or placebo for 14 days. For individual subject data, solid lines represent Example 1-treated and broken or dotted lines represent placebo-treated.

FIG. 6 shows inhibition of ear skin malonyl-CoA levels in Syrian hamsters treated with an orally administered (Example 8, 100 mg/kg) and topically administered ACC inhibitor (Example 3, 100 mg/ml).

FIG. 7 shows the percent contribution of DNL to sebum and circulating lipids (triglycerides) over time in the Syrian Hamster.

FIG. 8 shows inhibition of de novo lipogenesis in ear skin and liver in male Syrian Gold hamsters treated with an orally administered (Example 8, 100 mg/kg) and topically administered ACC inhibitor (Example 3, 100 mg/ml).

FIG. 9 shows the impact of chronic (19 days) once daily treatment with an orally administered ACC inhibitor (Example 8, 30 mg/kg) or vehicle on ear skin triglyceride levels in Syrian hamsters.

### DETAILED DESCRIPTION OF THE INVENTION

ACC, which catalyzes the conversion of acetyl-CoA to malonyl-CoA, plays a key role in regulating lipid metabolism. ACC is an essential and rate-limiting step in the de novo synthesis of fatty acids and regulates the oxidation of long chain fatty acids. The terms “de novo lipogenesis”, “DNL”, and “de novo fatty acid synthesis” are used to address the synthesis of fatty acids from non-lipid based sources. There are two closely related isoforms, ACC1 and ACC2. ACC inhibition has been of interest as a potential mechanism to treat type 2 diabetes mellitus and obesity (Harwood, 2005; Corbett 2009).

ACC Inhibitors Produce Morphological Changes in Sebaceous Glands in Rats and Dogs Consistent with Reduced Sebum Content.

In the course of preclinical in vivo studies in rats and dogs, it was discovered that multiple ACC inhibitors induced microscope morphologic changes in sebocytes consistent with reduced lipid/sebum content of sebaceous glands. Based on these observations, it was hypothesized that the ACC inhibitors may be reducing sebum lipid production in rats and dogs by inhibiting the de novo synthesis of fatty acids. Sebum is a complex mixture of lipids, comprised of triglycerides (30 to 50%), wax esters (26% to 30%), free fatty acids (15 to 30%), squalene (12 to 20%), cholesterol esters (3% to 6%) and free cholesterol (1.5 to 2.5%) (Ottaviani et al., 2010). Of these lipid classes, triglycerides, wax esters, free fatty acids and cholesterol esters all contain or are comprised of fatty acids. Elevated rates of sebum production are linked to both the onset and severity of acne (Janiczek-Dolphin et al., 2010). While it is known that human sebaceous glands are capable of de novo fatty acid synthesis (Downie and Kealey, 1998), the relative importance of this pathway within the sebocyte versus the use of exogenous circulating fatty acids for sebum biosynthesis was unknown.

### Sebum Biosynthesis in Humans is Highly Dependent on Sebocyte DNL

To elucidate the quantitative importance of de novo fatty acid synthesis to sebum production in humans, a clinical isotopic labeling study was performed. Mass isotopomer distribution analysis (MIDA) is a technique for measuring the synthesis of biological polymers and has been used for measuring the synthesis of lipids, carbohydrates, and proteins (reviewed by Hellerstein and Neese, 1999). The method involves introduction of a stable isotope-labeled precursor and quantifying the relative abundances of molecular species of a polymer differing only in mass (mass isotopomers) using mass spectrometry. De novo palmitate synthesis was calculated from the incorporation of the administered deuterium into palmitate fatty acid methyl ester measured by gas chromatography/mass spectrometry as described (Jones, 1996; Lee et al, 1994). The proportion of deuterium-labeled palmitate in the isolated lipids was used to calculate the fractional contribution of DNL to the total palmitate pool.

Subjects were male or female healthy volunteers between the ages of 18 and 49 years. The total duration of the study was 14 days. Subjects received an oral loading dose of deuterium labeled water (²H₂O) on day 1 to achieve up to ˜1.5% enrichment of the total body water pool. Subjects continued to take oral doses of ²H₂O once daily until Day 14 to maintain this enrichment at a steady state. Sebum was collected from the skin on the forehead and cheeks of subjects faces using Sebutape® (Kligman et al., 1986) on Days 4, 7, 11 and 14 to enable approximation of the steady state contribution of DNL to the sebum lipid pool. In addition, plasma was collected from each subject on Day 4, Day 7, Day 11 and Day 14 for measurement of deuterated water enrichment and assessment of the contribution of DNL to circulating lipids (i.e. palmitate in VLDL). Comparison of the contribution of DNL to circulating lipids vs. sebum was used to elucidate the importance of de novo fatty acid synthesis within the sebaceous glands. The contribution of DNL to lipid pools where circulating fatty acids are used as the principal source for complex lipid biosynthesis should mirror the contribution of DNL to circulating fatty acids. In contrast, the contribution of DNL to the lipid pool should be higher than the contribution to circulating lipids in cases where organ specific DNL plays a significant role.

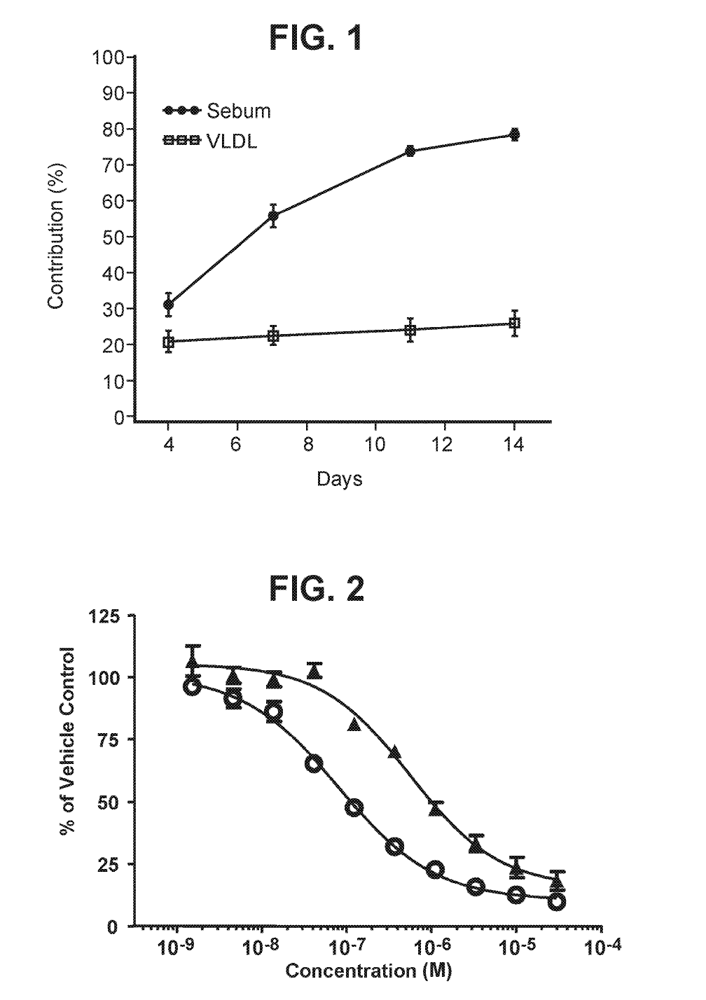

In contrast to other human lipid pools (Hellerstein, 1999), human sebum production was found to be highly dependent on de novo fatty acid synthesis, with approximately 80% of fatty acids contained in sebum derived from this pathway (FIG. 1). In addition, the contribution of DNL to sebum lipids was markedly greater than the contribution of DNL to circulating lipids (VLDL) (FIG. 1), demonstrating that DNL within the sebaceous gland is a major contributor to sebum biosynthesis in humans. This observation was unanticipated for two reasons. Firstly, DNL was believed to play a minor role in contributing to lipid homeostasis in humans (Hellerstein, 1999) and secondly, DNL within the sebocyte is of minor importance for sebum biosynthesis in the most commonly used preclinical model for sebum production, the Syrian hamster ear skin model (vida infra).

### ACC Inhibitors Suppress Human Sebocyte Cell DNL In Vitro

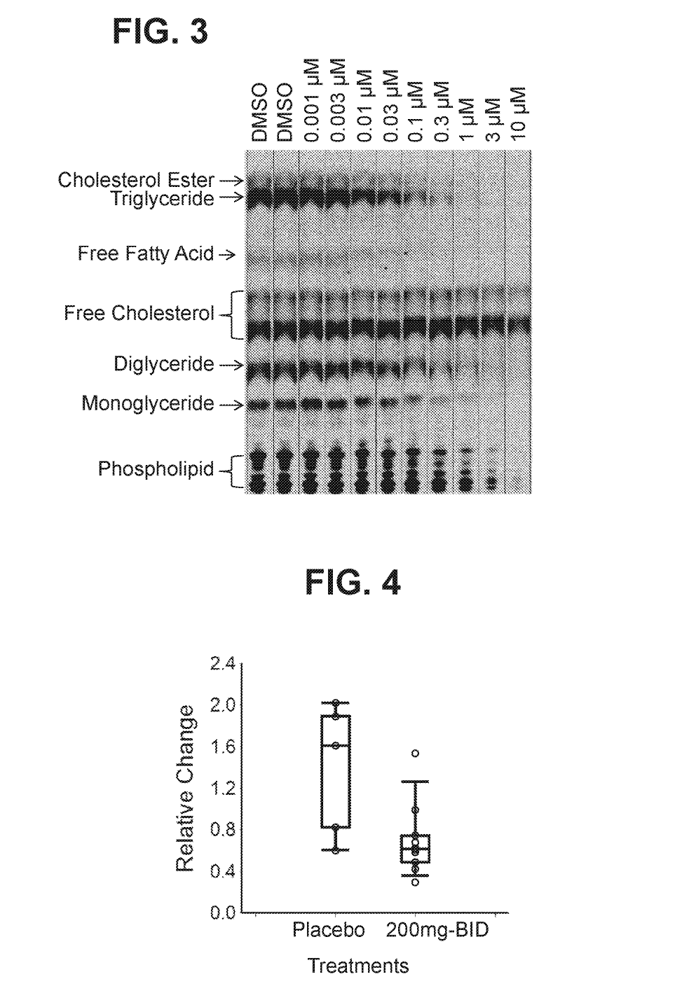

Elevated rates of sebum production are well correlated with the severity of acne vulgaris (Zouboulis, 2004). Treatments that suppress sebum production have been shown to reduce acne lesions in direct proportion to the measured reductions of sebum (Janiczek-Dolphin, 2010). The novel biology data demonstrating the high fractional contribution of DNL to sebum lipids suggested that agents that could suppress DNL may reduce sebum biosynthesis. To evaluate the ability of ACC inhibition to modulate DNL in human sebocytes, the impact of multiple ACC inhibitors to suppress ¹⁴C-acetate incorporation into sebum lipids was evaluated in SZ95 human sebocyte cells (Zouboulis et al., 1999). Example 1 is a selective dual ACC1/ACC2 inhibitor that dose-dependently suppressed DNL in cells, preclinical species and healthy human volunteers. FIG. 2 shows the effect of Example 1 and Example 3, relative to vehicle, on inhibition of DNL in human SZ95 sebocyte cells. FIG. 3 illustrates the effect of Example 3 vs. vehicle on suppressing incorporation of ¹⁴C-acetate into sebum lipid species in human SZ95 sebocte cells. Lipid species were separated by thin layer chromatography. Example 3 produced clear inhibition of incorporation of ¹⁴C-actetate into SZ95 cell lipid species containing or comprised of fatty acids (cholesterol esters, triglycerides, free fatty acids, diglycerides, monoglyceride and phospholipid) but did not alter incorporation of ¹⁴C-actetate into free cholesterol, which is not dependent on de novo fatty acid synthesis, demonstrating the specificity of the mechanism of action of Example 3 (FIG. 3).

Multiple other ACC inhibitors were evaluated in the SZ95 human sebocyte cell line. Table 1 summarizes the suppression of DNL with ACC inhibitors in this human sebocyte cell line. This assay may have utility in identifying ACC inhibitors likely to inhibit sebum production in vivo. A one-to-one correlation between the enzyme IC₅₀ₛ and sebocyte DNL EC₅₀ₛ are not necessarily expected due to difference in physical properties which may impact cell permeability, protein binding, and/or lipid solubility.

### Example 1

### Inhibits Sebum Production in Healthy Human Volunteers

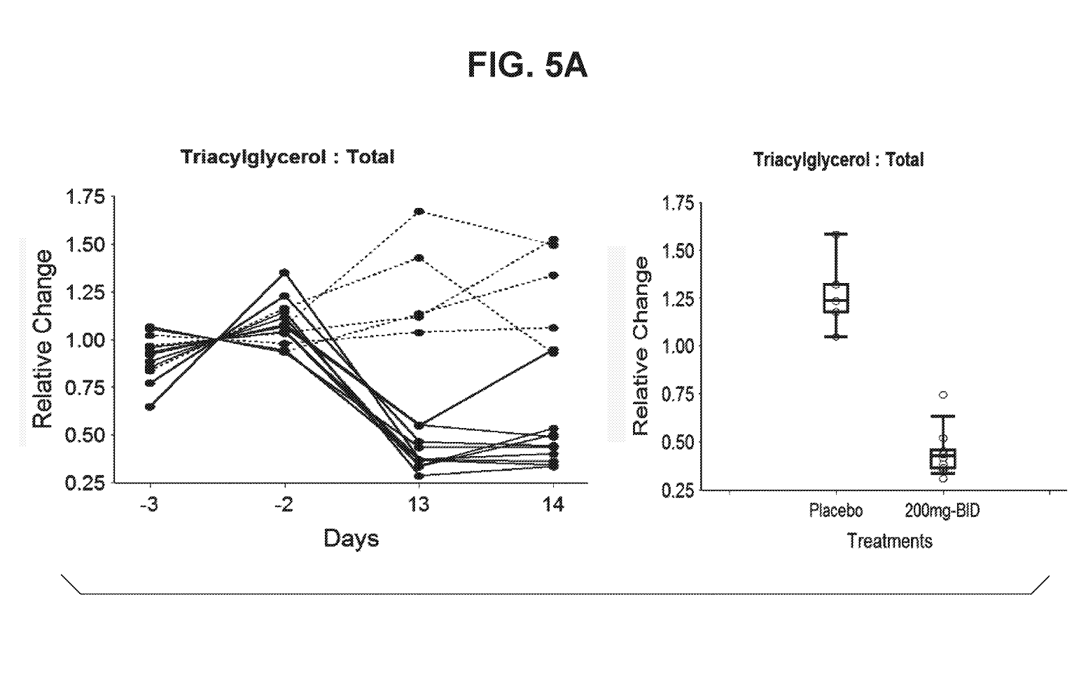

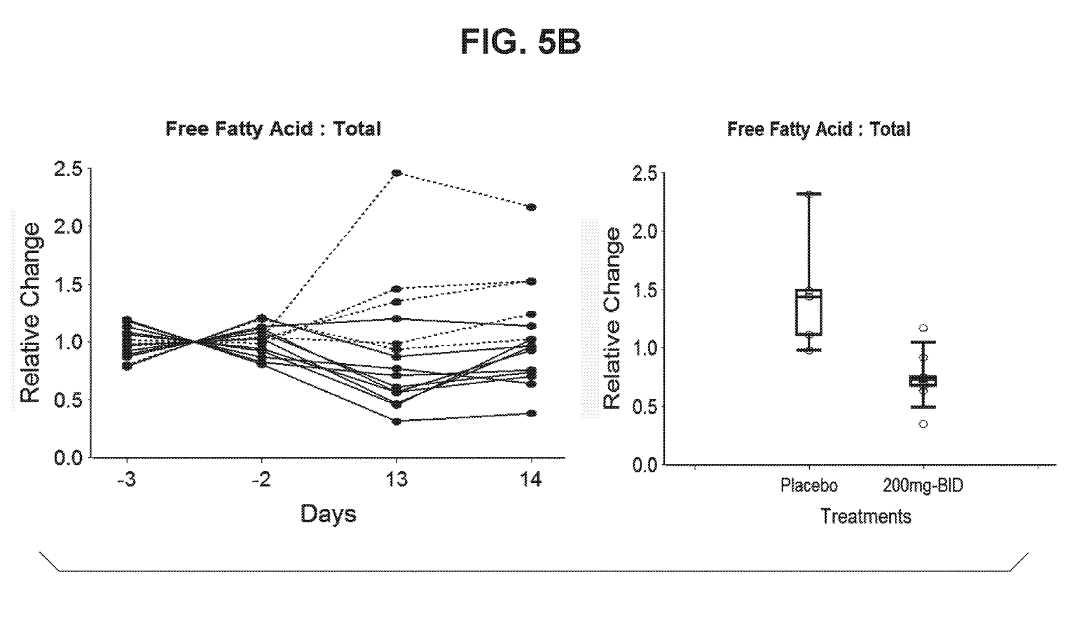

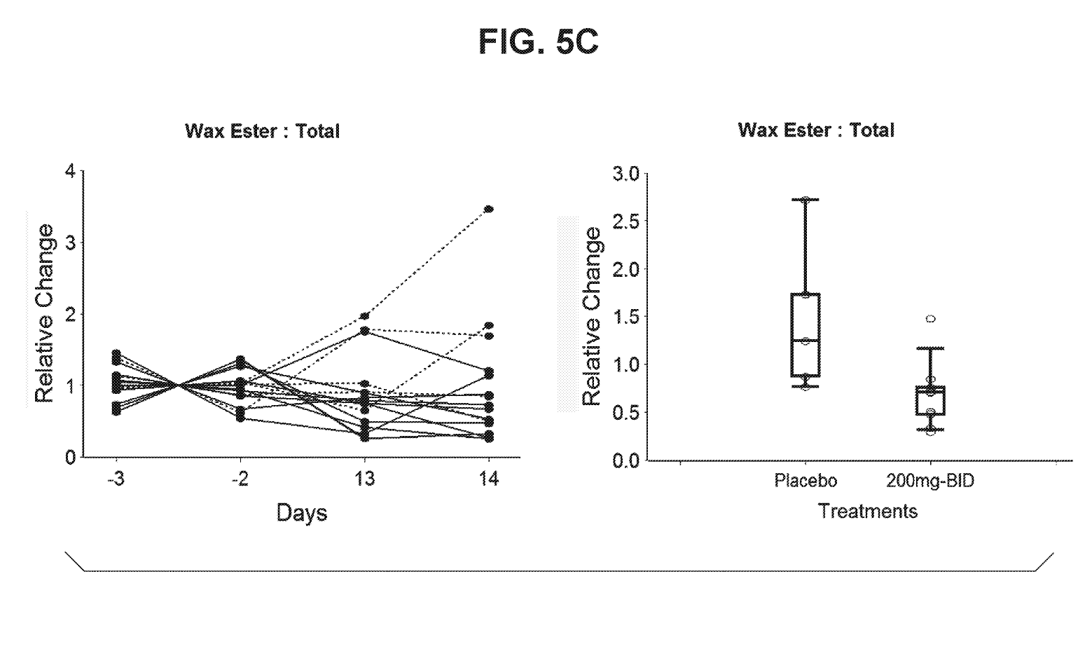

Sebum production was assessed in healthy human volunteers treated with 200 mg BID of Example 1 or placebo (PBO). This dose of Example 1 reduced production of sebum as measured by Sebumeter® (Courage+Khazaka electronic GmbH, Cologne, Germany) by 49% from baseline (PBO adjusted) in healthy volunteers (FIG. 4). Analysis of specific lipid classes from sebum collected at baseline and at end of study using Sebutape®, demonstrated that sebum triglycerides, the major lipid class in sebum, were decreased by 66% (PBO adjusted) (FIG. 5). Levels of sebum free fatty acids and wax esters, which are also dependent on DNL, were also reduced in Example 1 treated subjects (FIG. 5). In contrast, free cholesterol (which is not dependent on DNL) showed no change relative to PBO (data not shown). Squalene levels, which are also not dependent on DNL, showed a 2.6-fold increase relative to PBO. As squalene is a minor component of sebum, levels of total sebum as assessed by Sebumeter® showed a clear reduction (FIG. 4) despite this increase in squalene. The observation that Example 1 selectively suppressed levels of sebum lipids that contain or are comprised of fatty acid species, which are highly dependent on DNL, but not sebum lipid species that are not dependent on DNL, demonstrates the specificity of the mechanism for inhibition of sebum production.

ACC inhibition presents a novel opportunity to treat acne by reducing sebum production based on the observations that (1) de novo synthesized fatty acids accounted for approximately 80% of the fatty acids in human sebum lipids, (2) approximately 75% to 95% of human sebum lipid (including triglyceride, free fatty acids, wax esters and cholesterol esters) contain or are comprised of fatty acids, (3) ACC activity is essential for the de novo synthesis of fatty acids, (4) ACC inhibitors suppress DNL in human derived SZ95 sebocyte cells, and (5) Example 1 reduces sebum production in human volunteers.

Effects of ACC Inhibition on Sebum Production in Humans not Anticipated from the Most Commonly Used Preclinical Model for Sebum Production, the Hamster Ear Skin Model

The Syrian hamster ear model (Plewig and Luderschmidt, 1977) is the most commonly used in vivo model to measure drug effects on sebaceous glands. This model is commonly used because the ventral side of the earlobe in the Syrian hamster has a high density of sebaceous glands. Further, there is presumed translation of this model to humans since these glands are structurally similar to human sebaceous follicles in that they are large and have an infundibulum, a sebaceous duct, multiple lobules, and a pilary unit which enters from below into the gland (Plewig and Luderschmidt, 1977). The model has also been validated with multiple agents including oral Accutane® (Geiger, 1995).

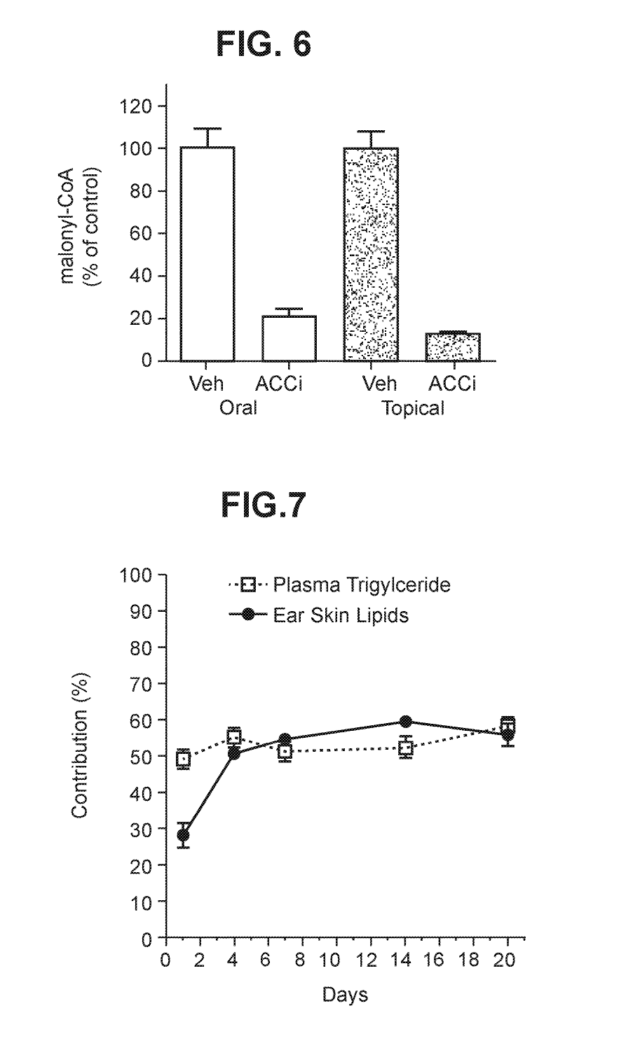

To assess the ability of ACC inhibitors to suppress ACC activity in this model, the effect of Example 8, administered by oral gavage, and Example 3, administered topically, to inhibit malonyl-CoA levels in ear skin was assessed one hour after a single treatment with compound (FIG. 6). Malonyl-CoA is the direct enzymatic product of ACC and measurement of malonyl-CoA has been used as a biomarker of ACC inhibition in vivo (Harwood et al., 2003; Glund et al., 2012; Freeman-Cook et al., 2012). Example 8, administered orally, and Example 3, administered topically suppressed hamster ear skin malonyl-CoA by 79% and 87% relative to vehicle treated animals. These observations demonstrate that orally administered Example 8 and the topically administered Example 3 each robustly inhibited ACC activity in this model.

To determine the importance of DNL within the sebaceous gland for sebum lipid biosynthesis in this model, ²H₂O was administered to enrich the water pool with deuterium. Plasma and ear skin were collected on days 1, 4, 7, 14 and 20. Comparison of the contribution of DNL to circulating lipids vs. skin lipids was used to elucidate the importance of de novo fatty acid synthesis within the Syrian hamster sebaceous glands. The contribution of DNL to lipid pools where circulating fatty acids are used as the principal source for complex lipid biosynthesis should mirror the contribution of DNL to circulating fatty acids. In contrast, in cases where organ specific DNL plays a significant role the contribution of DNL to the lipid pool should be higher than the contribution to circulating lipids. While the % contribution of DNL to sebum lipids at steady state was moderately high (55-60%) in this model, the contribution of DNL to sebum was similar to circulating triglycerides indicating that Syrian hamster sebaceous glands, in contrast to human sebaceous glands, predominantly utilize circulating fatty acids, rather than fatty acids synthesized within the sebocyte for sebum biosynthesis.

To further probe this hypothesis, the effect of orally administered Example 8 vs. vehicle and topically administered Example 3 vs. vehicle on inhibition of incorporation of DNL derived fatty acids into sebum was examined in the Syrian Hamster model. Each of these compounds was found to robustly inhibit ACC activity in skin (as assessed by ear skin malonyl-CoA levels) at the doses tested (FIG. 6). The topically administered compound would be anticipated to inhibit DNL only at the site of application, and not systemically while the orally administered compound would be expected to inhibit DNL systemically. Consequently, if the DNL derived fatty acids found in sebum were synthesized within the sebaceous gland, both the orally and topically administered compound would be expected to suppress incorporation of DNL derived fatty acids into sebum. In contrast, if the DNL derived fatty acids were synthesized in other lipogenic organs (e.g. liver) and delivered to the sebaceous gland via circulation, only the orally administered compound, but not the topically applied compound, would be anticipated to reduce incorporation of DNL derived fatty acids into sebum.

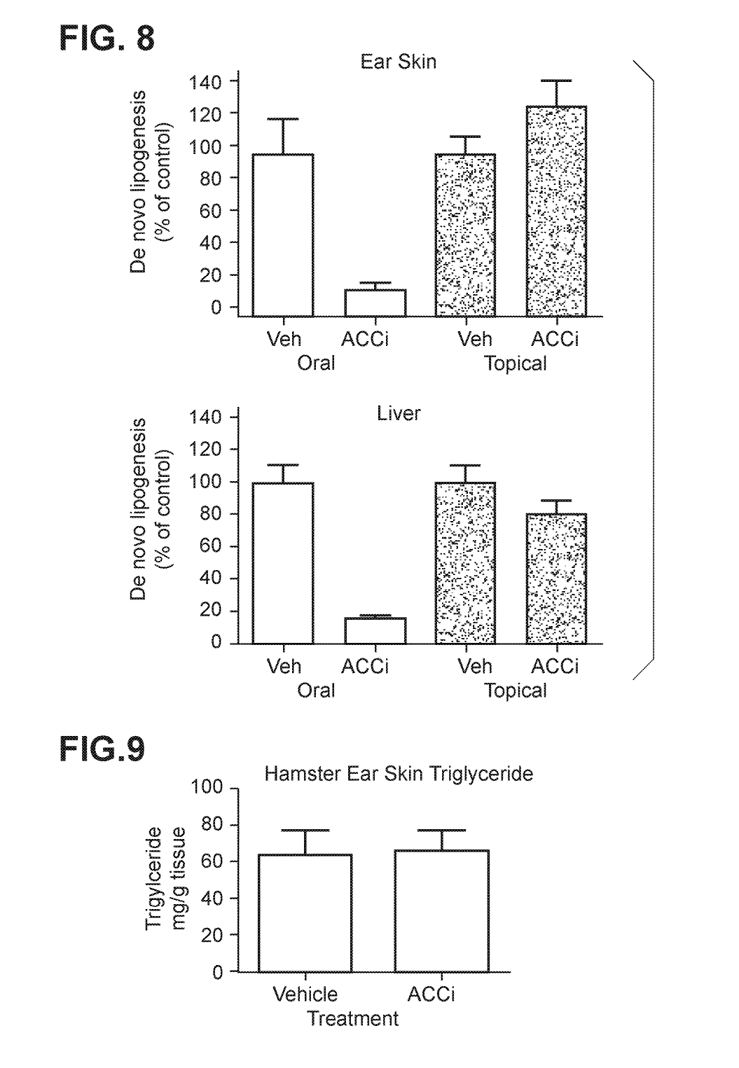

Syrian hamsters were treated with a single dose of orally administered Example 8 and topically administered Example 3 and then treated with an IP bolus of ¹⁴C-labeled acetate. The effect of each compound to suppress incorporation of DNL-derived fatty acids into sebum lipids was compared. In addition, impact of the compounds on DNL in the liver was also assessed. Orally administered Example 8 suppressed incorporation of de novo synthesized fatty acids in both skin and liver by 84% and 85% respectively (FIG. 8). In contrast, topically administered Example 3 failed to inhibit incorporation of de novo synthesized fatty acids in either skin or liver (FIG. 8).

As both compounds were shown to inhibit ACC activity, as assessed by malonyl-CoA production, by greater than ≧79% in skin, these results strongly imply that the de novo derived fatty acids in sebum lipids were synthesized elsewhere and delivered to the sebaceous gland via circulation. The ability of the orally administered Example 8, but not the topically administered Example 3, to inhibit DNL in the liver is also consistent with this hypothesis. These observations, taken together, are consistent with the hypothesis that, in contrast to humans, the Syrian Hamster predominantly utilizes circulating fatty acids for sebum production rather than fatty acids synthesized within the sebaceous gland.

The findings described above would suggest that the Syrian Hamster does not accurately predict human sebum lowering efficacy for the ACC mechanism as a result of the differences in the importance of sebaceous gland DNL for sebum biosynthesis. Since in humans approximately 80% of sebum fatty acids are derived from DNL and this DNL appears to occur largely in the sebaceous gland, administration of an ACC inhibitor orally or topically would suppress DNL in the sebaceous gland leading to reductions in sebum production. In contrast, in the Syrian hamster, which relies on circulating fatty acids rather than fatty acids synthesized in the sebaceous gland for sebum biosynthesis, would not show inhibition of sebum production through direct action on the sebocyte in vivo with either oral or topical ACC inhibitor treatment.

To test this hypothesis, Syrian hamsters were treated with orally administered Example 8 or vehicle once daily for 19 days. No changes in ear skin triglyceride levels were observed between ACC inhibitor-treated or vehicle-treated animals. This observation stands in contrast to the observation that human subjects treated with Example 1 show a 66% reduction in sebum triglycerides.

The differences in the importance of DNL within the sebaceous gland and striking difference in the impact of ACC inhibitors on suppression of sebum biosynthesis between humans and the Syrian hamster illustrate that the most commonly used preclinical model to assess novel sebum lowering mechanisms would have not identified the benefit of ACC inhibition.

In another embodiment, the present invention relates to a method of treating and/or preventing acne in a patient comprising the step of administering to the patient in need of such treatment a therapeutically effective amount of a compound of Formula (I), or a pharmaceutically acceptable salt thereof,

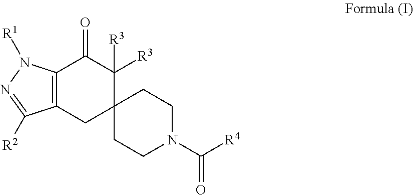

wherein

R¹ is (C₁-C₆)alkyl, (C₃-C₇)cycloalkyl, tetrahydrofuranyl or oxetanyl; wherein said (C₁-C₆)alkyl is optionally substituted with 1 to 2 substituents independently selected from (C₁-C₃)alkoxy; hydroxy, halo, phenyl, tetrahydrofuranyl or oxetanyl;

R² is hydrogen, halo, (C₁-C₃)alkyl, cyano or —C(═NH)(OCH₃);

R³ are each independently hydrogen or (C₁-C₃)alkyl;

R⁴ is (C₆-C₁₀)aryl, 5 to 12 membered heteroaryl or 8 to 12 membered fused heterocyclicaryl; wherein said (C₆-C₁₀)aryl, 5 to 12 membered heteroaryl or 8 to 12 membered fused heterocyclicaryl are each optionally substituted with one to three substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl.

In another embodiment, the present invention relates to a method of treating and/or preventing acne in a patient comprising the step of administering to the patient in need of such treatment a therapeutically effective amount of a compound of Formula (I) or a pharmaceutically acceptable salt thereof.

The present invention relates to a method of treating and/or preventing acne in patients comprising the step of administering orally to patients in need of such treatment a therapeutically effective amount of a compound of Formula (I) or a pharmaceutically acceptable salt thereof.

The present invention relates to a method of treating and/or preventing acne in patients comprising the step of administering topically to patients in need of such treatment a therapeutically effective amount of a compound of Formula (I) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (I), or a pharmaceutically acceptable salt thereof, and at least one pharmaceutically acceptable carrier.

In another embodiment, the present invention relates to methods of reducing sebum triglycerides, sebum free fatty acids, cholesterol esters and sebum waxy esters in patients comprising the step of administering to patients in need of such treatment a therapeutically effective amount of a compound of Formula (I) or a pharmaceutically acceptable salt thereof.

In another embodiment, the present invention relates to a method of treating and/or preventing acne in a patient comprising the step of administering to the patient in need of such treatment a therapeutically effective amount of 1-isopropyl-1′-(2-methyl-1H-benzo[d]imidazole-5-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-isopropyl-1′-(2-methyl-1H-benzo[d]imidazole-5-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-isopropyl-1′-(2-methyl-1H-benzo[d]imidazole-5-carbonyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount 1-isopropyl-1′-(2-methyl-1H-benzo[d]imidazole-5-carbonyl)-4,6-dihydrospiro[indazole-5, 4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-isopropyl-1′-(2-methyl-1H-benzo[d]imidazole-5-carbonyl)-4,6-dihydrospiro[indazole-5,4′-piperidin]-7(1H)-one or a pharmaceutically acceptable salt thereof.

In another embodiment, the present invention relates to a method of treating and/or preventing acne in a patient comprising the step of administering to the patient in need of such treatment a therapeutically effective amount of 1′-(1H-indazole-5-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1′-(1H-indazole-5-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1′-(1H-indazole-5-carbonyl)-1-isopropyl-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1′-(1H-indazole-5-carbonyl)-1-isopropyl-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1′-(1H-indazole-5-carbonyl)-1-isopropyl-4,6-dihydrospiro[indazole-5,4′-piperidin]-7(1H)-one or a pharmaceutically acceptable salt thereof.

In another embodiment, the present invention relates to a method of treating and/or preventing acne in a patient comprising the step of administering to the patient in need of such treatment a therapeutically effective amount of 1-isopropyl-1′-(1H-pyrrolo[3,2-b]pyridine-6-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-isopropyl-1′-(1H-pyrrolo[3,2-b]pyridine-6-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-isopropyl-1′-(1H-pyrrolo[3,2-b]pyridine-6-carbonyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1-isopropyl-1′-(1H-pyrrolo[3,2-b]pyridine-6-carbonyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-isopropyl-1′-(1H-pyrrolo[3,2-b]pyridine-6-carbonyl)-4,6-dihydrospiro[indazole-5,4′-piperidin]-7(1H)-one or a pharmaceutically acceptable salt thereof.

In another embodiment, the present invention relates to a method of treating and/or preventing acne in a patient comprising the step of administering to the patient in need of such treatment a therapeutically effective amount of 1-(tert-butyl)-1′-(1H-indazole-6-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-(tert-butyl)-1′-(1H-indazole-6-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-(tert-butyl)-1′-(1H-indazole-6-carbonyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1-(tert-butyl)-1′-(1H-indazole-6-carbonyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-(tert-butyl)-1′-(1H-indazole-6-carbonyl)-4,6-dihydrospiro[indazole-5,4′-piperidin]-7(1H)-one or a pharmaceutically acceptable salt thereof.

In another embodiment, the present invention relates to a method of treating and/or preventing acne in a patient comprising the step of administering to the patient in need of such treatment a therapeutically effective amount of 5-(1-isopropyl-7-oxo-1,4,6,7-tetrahydrospiro[indazole-5,4′-piperidin]-1′-ylcarbonyl)-1H-indazole-3-carbonitrile or pharmaceutically acceptable salt thereof.

In another embodiment, the present invention relates to a method of treating and/or preventing acne in a patient comprising the step of administering orally to the patient in need of such treatment a therapeutically effective amount of 5-(1-isopropyl-7-oxo-1,4,6,7-tetrahydrospiro[indazole-5,4′-piperidin]-1′-ylcarbonyl)-1H-indazole-3-carbonitrile or pharmaceutically acceptable salt thereof.

In another embodiment, the present invention relates to a method of treating and/or preventing acne in a patient comprising the step of administering topically to the patient in need of such treatment a therapeutically effective amount of 5-(1-isopropyl-7-oxo-1,4,6,7-tetrahydrospiro[indazole-5,4′-piperidin]-1′-ylcarbonyl)-1H-indazole-3-carbonitrile or pharmaceutically acceptable salt thereof.

The present invention relates to a method of treating and/or preventing acne in patients comprising the step of administering to patients in need of such treatment a pharmaceutical composition comprising a therapeutically effective amount of 5-(1-isopropyl-7-oxo-1,4,6,7-tetrahydrospiro[indazole-5,4′-piperidin]-1′-ylcarbonyl)-1H-indazole-3-carbonitrile, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 5-(1-isopropyl-7-oxo-1,4,6,7-tetrahydrospiro[indazole-5,4′-piperidin]-1′-ylcarbonyl)-1H-indazole-3-carbonitrile or a pharmaceutically acceptable salt thereof.

In another embodiment, the present invention relates to a method of treating and/or preventing acne in a patient comprising the step of administering to the patient in need of such treatment a therapeutically effective amount of 1-isopropyl-1′-(1H-pyrrolo[3,2-b]pyridine-2-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-isopropyl-1′-(1H-pyrrolo[3,2-b]pyridine-2-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-isopropyl-1′-(1H-pyrrolo[3,2-b]pyridine-2-carbonyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1-isopropyl-1′-(1H-pyrrolo[3,2-b]pyridine-2-carbonyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-isopropyl-1′-(1H-pyrrolo[3,2-b]pyridine-2-carbonyl)-4,6-dihydrospiro[indazole-5,4′-piperidin]-7(1H)-one or a pharmaceutically acceptable salt thereof.

The following compounds of Formula (I): 1′-(1H-indazole-5-carbonyl)-1-isopropyl-4,6-dihydrospiro[indazole-5,4′-piperidin]-7(1H)-one; 1-isopropyl-1′-(1H-pyrrolo[3,2-b]pyridine-6-carbonyl)-4,6-dihydrospiro[indazole-5,4′-piperidin]-7(1H)-one; 1-(tert-butyl)-1′-(1H-indazole-6-carbonyl)-4,6-dihydrospiro[indazole-5,4′-piperidin]-7(1H)-one; 5-(1-isopropyl-7-oxo-1,4,6,7-tetrahydrospiro[indazole-5,4′-piperidin]-1′-ylcarbonyl)-1H-indazole-3-carbonitrile; and 1-isopropyl-1′-(1H-pyrrolo[3,2-b]pyridine-2-carbonyl)-4,6-dihydrospiro[indazole-5,4′-piperidin]-7(1H)-one; may be prepared as described in U.S. Pat. No. 8,288,405, herein incorporated by reference.

In another embodiment, the present invention relates to a method of treating and/or preventing acne in a patient comprising the step of administering to the patient in need of such treatment a therapeutically effective amount of a compound of Formula (II), or a pharmaceutically acceptable salt thereof,

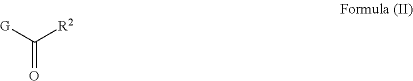

wherein

G is

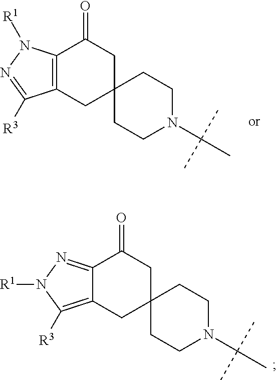

R¹ is a (C₁-C₆)alkyl or (C₃-C₇) cycloalkyl;

R² is indolyl, indazolyl, pyrrolopyridinyl, pyrazolopyridinyl, quinolinyl or benzoimidazolyl; wherein each R² group is optionally substituted with one to two substituents independently selected from a cyano, -L-C(O)NR⁴R⁵, -L-NR⁴R⁵, (C₁-C₃)alkyl, (C₁-C₃)alkoxy and halo;

R³ is hydrogen or (C₁-C₃)alkyl; L is a direct bond or —X(C₁-C₃)alkylene;

X is a direct bond, O or S;

R⁴ and R⁵ are each independently hydrogen, (C₁-C₃)alkyl, (C₃-C₇)cycloalkyl or four to seven membered heterocyclyl wherein said (C₁-C₃)alkyl, (C₃-C₇)cycloalkyl or four to seven membered heterocyclyl is optionally substituted with one to three fluoro or (C₁-C₃)alkoxy.

In another embodiment, the present invention relates to a method of treating and/or preventing acne in a patient comprising the step of administering to the patient in need of such treatment a therapeutically effective amount of a compound of Formula (II) or a pharmaceutically acceptable salt thereof.

The present invention relates to a method of treating and/or preventing acne in patients comprising the step of administering orally to patients in need of such treatment a therapeutically effective amount of a compound of Formula (II) or a pharmaceutically acceptable salt thereof.

The present invention relates to a method of treating and/or preventing acne in patients comprising the step of administering topically to patients in need of such treatment a therapeutically effective amount of a compound of Formula (II) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (II), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (II) or a pharmaceutically acceptable salt thereof.

In another embodiment, the present invention relates to a method of treating and/or preventing acne in a patient comprising the step of administering to the patient in need of such treatment a therapeutically effective amount of 1-isopropyl-1′-(6-methoxyquinoline-3-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-isopropyl-1′-(6-methoxyquinoline-3-carbonyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-isopropyl-1′-(6-methoxyquinoline-3-carbonyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1-isopropyl-1′-(6-methoxyquinoline-3-carbonyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-isopropyl-1′-(6-methoxyquinoline-3-carbonyl)-4,6-dihydrospiro[indazole-5,4′-piperidin]-7(1H)-one or a pharmaceutically acceptable salt thereof. 1-isopropyl-1′-(6-methoxyquinoline-3-carbonyl)-4,6-dihydrospiro[indazole-5,4′-piperidin]-7(1H)-one

In another embodiment, the present invention relates to a method of treating and/or preventing acne in a patient comprising the step of administering to the patient in need of such treatment a therapeutically effective amount of 1′-(2-(tert-butylamino)quinoline-7-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1′-(2-(tert-butylamino)quinoline-7-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1′-(2-(tert-butylamino)quinoline-7-carbonyl)-1-isopropyl-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1′-(2-(tert-butylamino)quinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1′-(2-(tert-butylamino)quinoline-7-carbonyl)-1-isopropyl-4,6-dihydrospiro[indazole-5,4′-piperidin]-7(1H)-one or a pharmaceutically acceptable salt thereof.

In another embodiment, the present invention relates to a method of treating and/or preventing acne in a patient comprising the step of administering to the patient in need of such treatment a therapeutically effective amount of 1′-(2-aminoquinoline-7-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1′-(2-aminoquinoline-7-carbonyl)-1-isopropyl-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1′-(2-aminoquinoline-7-carbonyl)-1-isopropyl-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in a patient comprising the step of administering to the patient in need of such treatment a therapeutically effective amount of 1′(2-aminoquinoline-7-carbonyl)-1-isopropyl-4,6-dihydrospiro[indazole-5,4′-piperidin]-7(1H)-one or a pharmaceutically acceptable salt thereof.

In another embodiment, the present invention relates to a method of reducing sebum triglycerides, sebum free fatty acids, cholesterol esters and sebum waxy esters in a patient comprising the step of administering to the patient in need of such treatment a pharmaceutical composition comprising a therapeutically effective amount of 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier.

In another embodiment, the present invention relates to the use of 1′-(2-aminoquinoline-7-carbonyl)-1-isopropyl-4,6-dihydrospiro[indazole-5,4′-piperidin]-7(1H)-one, or a pharmaceutically acceptable salt thereof, in the manufacture of a medicament for the treatment of acne.

In another embodiment, the present invention relates to the use of a pharmaceutical composition comprising 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier thereof in the manufacture of a medicament for the treatment of acne.

In another embodiment, the present invention relates to the use of 1′-(2-aminoquinoline-7-carbonyl)-1-isopropyl-4,6-dihydrospiro[indazole-5,4′-piperidin]-7(1H)-one, or a pharmaceutically acceptable salt thereof, in the manufacture of a medicament for reducing sebum triglycerides, sebum free fatty acids, cholesterol esters and sebum waxy esters in a patient.

In another embodiment, the present invention relates to the use of a pharmaceutical composition comprising 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier thereof in the manufacture of a medicament for reducing sebum triglycerides, sebum free fatty acids, cholesterol esters and sebum waxy esters in a patient.

The following Formula (II) compounds: 1-isopropyl-1′-(6-methoxyquinoline-3-carbonyl)-4,6-dihydrospiro[indazole-5,4′-piperidin]-7(1H)-one; 1′-(2-(tert-butylamino)quinoline-7-carbonyl)-1-isopropyl-4,6-dihydrospiro[indazole-5,4′-piperidin]-7(1H)-one; and 1′-(2-aminoquinoline-7-carbonyl)-1-isopropyl-4,6-dihydrospiro[indazole-5,4′-piperidin]-7(1H)-one; may be prepared using similar procedures as described in U.S. Pat. No. 8,288,405, US 2012/0108619 herein incorporated by reference.

In another embodiment, the present invention relates to a method of treating and/or preventing acne in a patient comprising the step of administering to the patient in need of such treatment a therapeutically effective amount of a compound of Formula (III), or a pharmaceutically acceptable salt thereof,

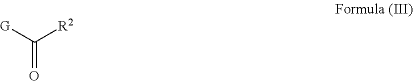

wherein G is

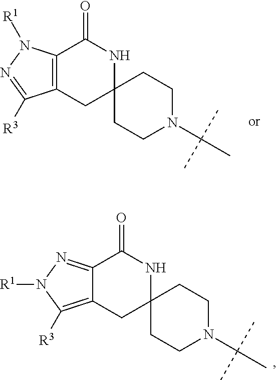

R¹ is a (C₁-C₆)alkyl or (C₃-C₅) cycloalkyl;

R² is phenyl, naphthyl, a 5 to 12 membered heteroaryl or a 8 to 12 membered fused heterocyclicaryl; wherein each R² group is optionally substituted with one to three substituents independently selected from (C₁-C₃)alkyl, (C₁-C₃)alkoxy halo and CONH₂; and

R³ is hydrogen or (C₁-C₃)alkyl; or a pharmaceutically acceptable salt thereof.

In another embodiment, the present invention relates to a method of treating and/or preventing acne in a patient comprising the step of administering to the patient in need of such treatment a therapeutically effective amount of a compound of Formula (III) or a pharmaceutically acceptable salt thereof.

The present invention relates to a method of treating and/or preventing acne in patients comprising the step of administering orally to patients in need of such treatment a therapeutically effective amount of a compound of Formula (III) or a pharmaceutically acceptable salt thereof.

The present invention relates to a method of treating and/or preventing acne in patients comprising the step of administering topically to patients in need of such treatment a therapeutically effective amount of a compound of Formula (III) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (III), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (III) or a pharmaceutically acceptable salt thereof.

In another embodiment, the present invention relates to a method of treating and/or preventing acne in a patient comprising the step of administering to the patient in need of such treatment a therapeutically effective amount of 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering orally to the patient in need of such treatment a therapeutically effective amount of 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering topically to the patient in need of such treatment a therapeutically effective amount of 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one or pharmaceutically acceptable salt thereof.

The present invention relates to a method of treating and/or preventing acne in patients comprising the step of administering to patients in need of such treatment a pharmaceutical composition comprising a therapeutically effective amount of 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one or a pharmaceutically acceptable salt thereof.

In another embodiment, the present invention relates to a method of treating and/or preventing acne in a patient comprising the step of administering to the patient in need of such treatment a therapeutically effective amount of 2′-(tert-butyl)-1-(8-methoxy-2-naphthoyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering orally to the patient in need of such treatment a therapeutically effective amount of 2′-(tert-butyl)-1-(8-methoxy-2-naphthoyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering topically to the patient in need of such treatment a therapeutically effective amount of 2′-(tert-butyl)-1-(8-methoxy-2-naphthoyl)-4′,6′-dihydrospiro[piperidine-4,5′-pyrazolo[3,4-c]pyridin]-7′(2′H)-one or pharmaceutically acceptable salt thereof.

The present invention relates to a method of treating and/or preventing acne in patients comprising the step of administering to patients in need of such treatment a pharmaceutical composition comprising a therapeutically effective amount of 2′-(tert-butyl)-1-(8-methoxy-2-naphthoyl)-4′,6′-dihydrospiro[piperidine-4,5′-pyrazolo[3,4-c]pyridin]-7′(2′H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 2′-(tert-butyl)-1-(8-methoxy-2-naphthoyl)-4′,6′-dihydrospiro[piperidine-4,5′-pyrazolo[3,4-c]pyridin]-7′(2′H)-one or a pharmaceutically acceptable salt thereof.

In another embodiment, the present invention relates to a method of treating and/or preventing acne in a patient comprising the step of administering to the patient in need of such treatment a therapeutically effective amount of 2′-(tert-butyl)-1-(2-methoxyquinoline-7-carbonyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering orally to the patient in need of such treatment a therapeutically effective amount 2′-(tert-butyl)-1-(2-methoxyquinoline-7-carbonyl)-4′,6′-dihydrospiro[piperidine-4,5′-pyrazolo[3,4-c]pyridin]-7′(2′H)-one or pharmaceutically acceptable salt thereof.

In another embodiment, the present invention relates to a method of treating and/or preventing acne in a patient comprising the step of administering topically to the patient in need of such treatment a therapeutically effective amount of 2′-(tert-butyl)-1-(2-methoxyquinoline-7-carbonyl)-4′,6′-dihydrospiro[piperidine-4,5′-pyrazolo[3,4-c]pyridin]-7′(2′H)-one or pharmaceutically acceptable salt thereof.

The present invention relates to a method of treating and/or preventing acne in patients comprising the step of administering to patients in need of such treatment a pharmaceutical composition comprising a therapeutically effective amount of 2′-(tert-butyl)-1-(2-methoxyquinoline-7-carbonyl)-4′,6′-dihydrospiro[piperidine-4,5′-pyrazolo[3,4-c]pyridin]-7′(2′H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 2′-(tert-butyl)-1-(2-methoxyquinoline-7-carbonyl)-4′,6′-dihydrospiro[piperidine-4,5′-pyrazolo[3,4-c]pyridin]-7′(2′H)-one or a pharmaceutically acceptable salt thereof.

The following compounds of Formula (III): 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one; 2′-(tert-butyl)-1-(2-methoxyquinoline-7-carbonyl)-4′,6′-dihydrospiro[piperidine-4,5′-pyrazolo[3,4-c]pyridin]-7′(2′H)-one; and 2′-(tert-butyl)-1-(8-methoxy-2-naphthoyl)-4′,6′-dihydrospiro[piperidine-4,5′-pyrazolo[3,4-c]pyridin]-7′(2′H)-one; may be prepared as described in US 2012/0108619, herein incorporated by reference.

In another embodiment, the present invention relates to a method of treating and/or preventing acne in a patient comprising the step of administering to the patient in need of such treatment a therapeutically effective amount of a compound of Formula (IV), or a pharmaceutically acceptable salt thereof,

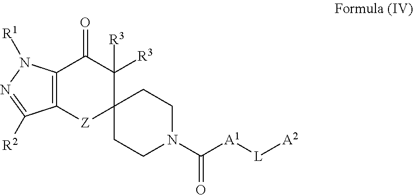

wherein

R¹ is (C₁-C₆)alkyl, (C₃-C₇)cycloalkyl, tetrahydrofuranyl or oxetanyl; wherein said (C₁-C₆)alkyl is optionally substituted with 1 to 3 substituents independently selected from (C₁-C₃)alkoxy, hydroxy, fluoro, phenyl, tetrahydrofuranyl or oxetanyl;

R² is hydrogen, halo, (C₁-C₃)alkyl, or cyano;

R³ are each independently hydrogen or (C₁-C₃)alkyl;

L is a direct bond or a (C₁-C₆)alkylene wherein one carbon of the (C₁-C₆)alkylene is optionally replaced by —C(O)—, —C(O)NH—, —NHC(O), O, S, NH or N(C₁-C₃)alkyl;

Z is CH₂ or O;

A¹ and A² are each independently (C₆-C₁₀)aryl, 5 to 12 membered heteroaryl or 8 to 12 membered fused heterocyclicaryl; wherein said (C₆-C₁₀)aryl, 5 to 12 membered heteroaryl or 8 to 12 membered fused heterocyclicaryl are each optionally substituted with one to three substituents independently selected from (C₁-C₃)alkyl, (C₁-C₃)alkoxy, halo, amino, (C₁-C₃)alkylamino, di(C₁-C₃)alkylamino, hydroxy, cyano and amido wherein the alkyl portion of the (C₁-C₃)alkyl, (C₁-C₃)alkoxy, (C₁-C₃)alkylamino and di(C₁-C₃)alkylamino are optionally substituted with one to five fluoro; and wherein one of A¹ or A² is substituted by CO₂R⁴, (C₁-C₆)CO₂R⁴, tetrazolyl or (C₁-C₆)tetrazolyl; and

R⁴ is (C₁-C₈)alkyl, (C₃-C₈)cycloalkyl or (C₁-C₆)alkyl-(C₃-C₈)cycloalkyl;

or a pharmaceutically acceptable salt thereof.

In another embodiment, the present invention relates to a method of treating and/or preventing acne in a patient comprising the step of administering to the patient in need of such treatment a therapeutically effective amount of a compound of Formula (IV) or a pharmaceutically acceptable salt thereof.

The present invention relates to a method of treating and/or preventing acne in patients comprising the step of administering orally to patients in need of such treatment a therapeutically effective amount of a compound of Formula (IV) or a pharmaceutically acceptable salt thereof.

The present invention relates to a method of treating and/or preventing acne in patients comprising the step of administering topically to patients in need of such treatment a therapeutically effective amount of a compound of Formula (IV) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (IV), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (IV) or a pharmaceutically acceptable salt thereof.

In another embodiment, the present invention relates to a method of treating and/or preventing acne in a patient comprising the step of administering to the patient in need of such treatment a therapeutically effective amount of a compound of Formula (V), or a pharmaceutically acceptable salt thereof,

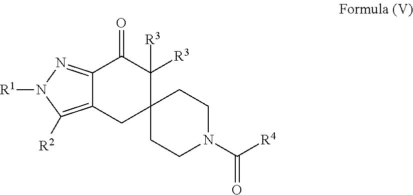

wherein

R¹ is (C₁-C₆)alkyl, (C₃-C₇)cycloalkyl, tetrahydrofuranyl or oxetanyl; wherein said (C₁-C₆)alkyl is optionally substituted with 1 to 2 substituents independently selected from (C₁-C₃)alkoxy; hydroxy, halo, phenyl, tetrahydrofuranyl or oxetanyl;

R² is hydrogen, halo, (C₁-C₃)alkyl, cyano or —C(═NH)(OCH₃);

R³ are each independently hydrogen or (C₁-C₃)alkyl;

R⁴ is (C₆-C₁₀)aryl, 5 to 12 membered heteroaryl or 8 to 12 membered fused heterocyclicaryl; wherein said (C₆-C₁₀)aryl, 5 to 12 membered heteroaryl or 8 to 12 membered fused heterocyclicaryl are each optionally substituted with one to three substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl; or a pharmaceutically acceptable salt thereof. A preferred embodiment of the present invention are compounds of Formula (I) wherein R⁴ is (C₆-C₁₀) aryl selected from phenyl or naphthyl; a 5 to 12 membered heteroaryl selected from pyridinyl, pyrazolyl, pyrimidinyl, triazolyl, indolizinyl, indazolyl, pyrrolo[2,3-b]pyridinyl, pyrrolo[3,2-b]pyridinyl, pyrrolo[1,2-a]pyrazinyl, imidazo[1,2-a]pyridinyl, imidazo[1,5-a]pyridinyl, benzo[d]imidazolyl, pyrazolo[3,4-b]pyridinyl, pyrazolo[4,3-b]pyridinyl, pyrazolo[1,5-a]pyrimidinyl, benzo[d]imidazol-2-onyl, 1,6-naphthyridinyl, quinoxalinyl, quinolin-4-onyl or isoquinolin-1-onyl; or an 8 to 12 membered fused heterocyclicaryl selected from 3,4-dihydroquinolin-2-onyl or indolin-2-onyl; wherein each R⁴ group is optionally substituted with one to four substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl; or a pharmaceutically acceptable salt thereof.

In another embodiment, the present invention relates to a method of treating and/or preventing acne in a patient comprising the step of administering to the patient in need of such treatment a therapeutically effective amount of a compound of Formula (V) or a pharmaceutically acceptable salt thereof.

The present invention relates to a method of treating and/or preventing acne in patients comprising the step of administering orally to patients in need of such treatment a therapeutically effective amount of a compound of Formula (V) or a pharmaceutically acceptable salt thereof.

The present invention relates to a method of treating and/or preventing acne in patients comprising the step of administering topically to patients in need of such treatment a therapeutically effective amount of a compound of Formula (V) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (V), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (V) or a pharmaceutically acceptable salt thereof.

In another embodiment, the present invention relates to a method of treating and/or preventing acne in a patient comprising the step of administering to the patient in need of such treatment a therapeutically effective amount of a compound of Formula (VI), or a pharmaceutically acceptable salt thereof,

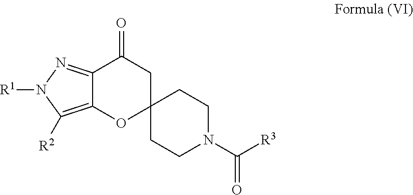

wherein

R¹ is (C₁-C₄)alkyl, (C₃-C₆)cycloalkyl, tetrahydrofuranyl, benzyl, pyridyl, or phenyl optionally substituted 1 to 2 substituents independently selected from cyano and methoxy (preferably, R¹ is (C₁-C₄)alkyl, (C₃-C₆)cycloalkyl, or tetrahydrofuranyl, more preferably, ethyl, isopropyl or t-butyl, most preferably, t-butyl);

R² is hydrogen, methyl or ethyl (preferably R² is hydrogen or methyl, more preferably hydrogen);

R³ is a chemical moiety selected from the group consisting of

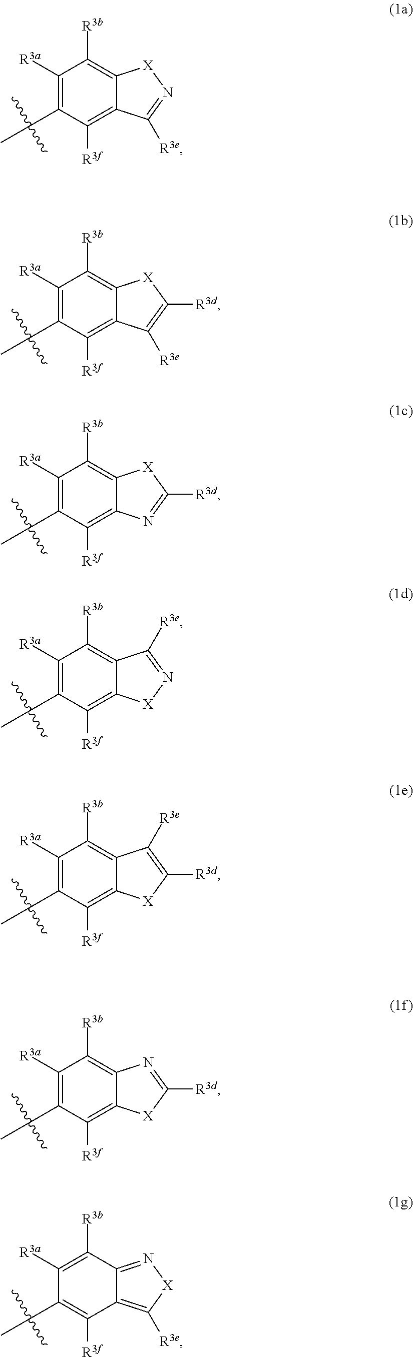

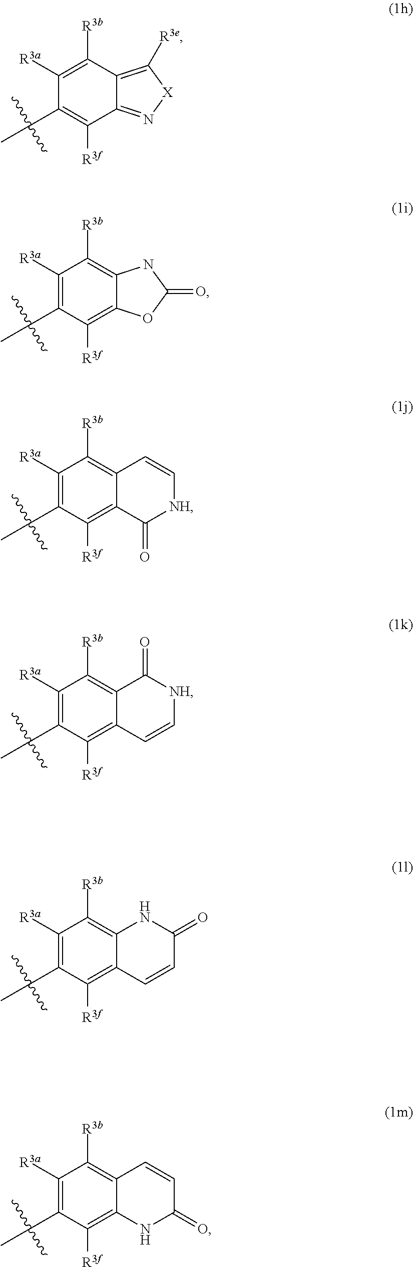

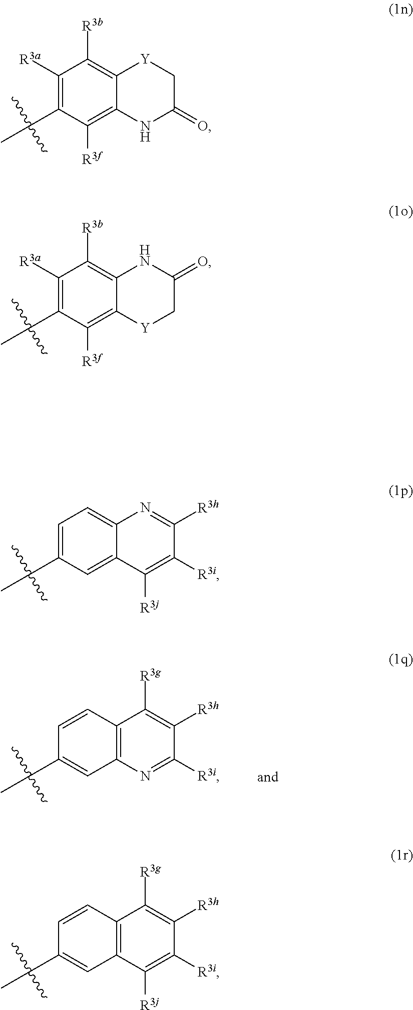

(preferably, R³ is a chemical moiety of formula (1a), (1c), (1d), (1f), (1i), (1j), (1k), (1l), (1m), (1n), (1o), (1p) or (1q), more preferably, formula (1a), (1c), (1d), (1f), (1j) or (1k);

where X is O, S, or N—R^{3c} (preferably, X is O or N—R^{3c}, more preferably, N—R^{3c});

Y is CH₂ or O (preferably, Y is CH₂);

R^{3a} is hydrogen or methyl (R^{3a} is preferably hydrogen);

R^{3b} is hydrogen, methyl, ethyl, halo, methoxy, or ethoxy (R^{3b} is preferably, hydrogen, methyl methoxy, chloro or fluoro, more preferably, when R³ is a chemical moiety of formula (1a), (1c), (1d), or (1f), then R^{3b} is hydrogen, methyl or chloro, and when R³ is a chemical moiety of formula (1b), (1e), (1g), (1h), (1i), (1j), (1k), (1m), (1n), or (1o), then R^{3b} is hydrogen);

R^{3c} is hydrogen, methyl, ethyl, or 3- to 6-membered cycloalkyl (preferably, R^{3c} is hydrogen or methyl);

R^{3d} is hydrogen, methyl, or hydroxyl (preferably, R^{3d} is hydrogen);

R^{3e} is hydrogen, methyl, ethyl, halo, or amino (preferably, R^{3e} is hydrogen or methyl, more preferably, hydrogen);

R^{3f} is hydrogen, methyl, or methoxy (preferably, R^{3f} is hydrogen);

R^{3g} is hydrogen, or methoxy (preferably, R^{3g} is hydrogen);

R^{3h} is hydrogen, methyl, methoxy, or halo (preferably, R^{3h} is hydrogen);

R³ⁱ is hydrogen, methyl, or methoxy (preferably, R³ⁱ is hydrogen); and

R^{3j} is hydrogen, or methoxy (preferably, R^{3j} is hydrogen).

In another embodiment, the present invention relates to a method of treating and/or preventing acne in a patient comprising the step of administering to the patient in need of such treatment a therapeutically effective amount of a compound of Formula (VI) or a pharmaceutically acceptable salt thereof.

The present invention relates to a method of treating and/or preventing acne in patients comprising the step of administering orally to patients in need of such treatment a therapeutically effective amount of a compound of Formula (VI) or a pharmaceutically acceptable salt thereof.

The present invention relates to a method of treating and/or preventing acne in patients comprising the step of administering topically to patients in need of such treatment a therapeutically effective amount of a compound of Formula (VI) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (VI), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (VI) or a pharmaceutically acceptable salt thereof.

In another embodiment, the present invention relates to a method of treating and/or preventing acne in a patient comprising the step of administering to the patient in need of such treatment a therapeutically effective amount of 2′-(tert-butyl)-1-(1H-indazole-5-carbonyl)-2′H-spiro[piperidine-4,5′-pyrano[3,2-c]pyrazol]-7′(6′H)-one or pharmaceutically acceptable salt thereof.

In another embodiment, the present invention relates to a method of treating and/or preventing acne in a patient comprising the step of administering orally to the patient in need of such treatment a therapeutically effective amount 2′-(tert-butyl)-1-(1H-indazole-5-carbonyl)-2′H-spiro[piperidine-4,5′-pyrano[3,2-c]pyrazol]-7′(6′H)-one or pharmaceutically acceptable salt thereof.

In another embodiment, the present invention relates to a method of treating and/or preventing acne in a patient comprising the step of administering topically to the patient in need of such treatment a therapeutically effective amount of 2′-(tert-butyl)-1-(1H-indazole-5-carbonyl)-2′H-spiro[piperidine-4,5′-pyrano[3,2-c]pyrazol]-7′(6′H)-one or pharmaceutically acceptable salt thereof.

The present invention relates to a method of treating and/or preventing acne in patients comprising the step of administering to patients in need of such treatment a pharmaceutical composition comprising a therapeutically effective amount of 2′-(tert-butyl)-1-(1H-indazole-5-carbonyl)-2′H-spiro[piperidine-4,5′-pyrano[3,2-c]pyrazol]-7′(6′H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 2′-(tert-butyl)-1-(1H-indazole-5-carbonyl)-2′H-spiro[piperidine-4,5′-pyrano[3,2-c]pyrazol]-7′(6′H)-one or a pharmaceutically acceptable salt thereof.

2′-(tert-Butyl)-1-(1H-indazole-5-carbonyl)-2′H-spiro[piperidine-4,5′-pyrano[3,2-c]pyrazol]-7′(6′H)-one may be prepared using the procedures described in WO 2009/144555 herein incorporated by reference.

In another embodiment, the present invention relates to a method of treating and/or preventing acne in a patient comprising the step of administering to the patient in need of such treatment a therapeutically effective amount of a compound of Formula (VII), or a pharmaceutically acceptable salt thereof,

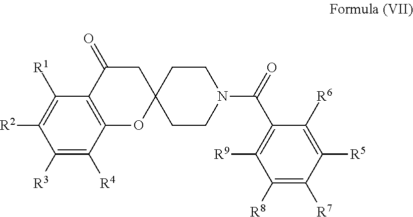

wherein:

R¹ is H, OH, halo, cyano, C₁₋₃ alkyl, C₁₋₃ alkoxy, C₁₋₃ haloalkyl, C₁₋₃ haloalkoxy, C₁₋₃ alkylsulfonyl-, —CO(O)H, —C(O)OC₁₋₃ alkyl or phenyl, wherein said phenyl is optionally substituted with one to five R¹⁰; each R¹⁰ is independently OH, halo, cyano, C₁₋₃ alkyl, C₁₋₃ alkoxy, C₁₋₃ haloalkyl or C₁₋₃ haloalkoxy;

R² and R³ are each independently H, OH, halo, cyano, C₁₋₃ alkyl, C₁₋₃ alkoxy, C₁₋₃ haloalkyl, C₁₋₃ haloalkoxy, C₁₋₃ alkylsulfonyl-, —CO(O)H, —C(O)OC₁₋₃ alkyl, —C(O)NR¹¹R¹², or phenyl wherein said phenyl is optionally substituted with one to five R¹⁰;

R¹¹ and R¹² are taken separately and are each independently H or C₁₋₃ alkyl, or R¹¹ and R¹² are taken together, with the nitrogen to which they are attached, to form a 4-7-membered heterocycloalkyl;

R⁴ is H, halo, cyano, C₁₋₃ alkyl or C₁₋₃ haloalkyl;

(f) R⁶ is taken separately and is H, OH, halo, C₁₋₃ alkyl, C₁₋₃ alkoxy, C₁₋₃ haloalkyl or C₁₋₃ haloalkoxy;

R⁷ is taken separately and is H, OH, halo, C₁₋₃ alkyl, C₁₋₃ alkoxy, C₁₋₃ haloalkyl or C₁₋₃ haloalkoxy;

R⁵ is taken separately and is a 4-7-membered heteroaryl optionally substituted with halo, C₁₋₃ alkyl, C₁₋₃ alkoxy, C₁₋₃ alkyl-OH, C₁₋₃ haloalkyl or C₁₋₃; or R⁵ is taken together with R⁶ or R⁷, and with the phenyl to which R⁵ and R⁶ or R⁷ are attached, to form a polycyclic heterocyclic radical, with a nitrogen-bearing ring wherein at least one nitrogen atom is bound to a carbon atom of said phenyl, wherein the nitrogen-bearing ring is optionally fused to cyclohexene, 5,6-dihydro-pyridine or 5,6-dihydro-1H-pyridin-2-one, and wherein the nitrogen-bearing ring is optionally substituted independently with one to two oxo, halo, C₁₋₃ alkyl, C₁₋₃ alkoxy, C₁₋₃ alkyl-OH, C₁₋₃ haloalkyl, C₁₋₃ haloalkoxy, 4-7-membered heteroaryl, 4-7-membered heterocycloalkyl or phenyl, wherein said phenyl is optionally substituted with one to five R¹⁰, provided that R⁵ is not taken together with R⁶ to form a benzotriazolyl or a benzooxadiazolyl and provided that R⁵ is not taken together with R⁷ to form a benzooxadiazolyl; and

R⁸ and R⁹ are independently H, OH, halo, C₁₋₃ alkyl, C₁₋₃ alkoxy, C₁₋₃ haloalkyl or C₁₋₃ haloalkoxy.

In another embodiment, the present invention relates to a method of treating and/or preventing acne in a patient comprising the step of administering to the patient in need of such treatment a therapeutically effective amount of a compound of Formula (VII) or a pharmaceutically acceptable salt thereof.

The present invention relates to a method of treating and/or preventing acne in patients comprising the step of administering orally to patients in need of such treatment a therapeutically effective amount of a compound of Formula (VII) or a pharmaceutically acceptable salt thereof.

The present invention relates to a method of treating and/or preventing acne in patients comprising the step of administering topically to patients in need of such treatment a therapeutically effective amount of a compound of Formula (VII) or a pharmaceutically acceptable salt thereof.

The present invention relates to a method of treating and/or preventing acne in patients comprising the step of administering to patients in need of such treatment a pharmaceutical composition comprising a therapeutically effective amount of a compound of Formula (VII), or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of a compound of Formula (VII) or a pharmaceutically acceptable salt thereof.

In another embodiment, the present invention relates to 1-isopropyl-1′-(7-methoxy-2-naphthoyl)-4,6-dihydrospiro[indazole-5,4′-piperidin]-7(1H)-one or a pharmaceutically acceptable salt thereof.

In another embodiment, the present invention relates to a pharmaceutical composition comprising 1-isopropyl-1′-(7-methoxy-2-naphthoyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of treating and/or preventing acne in a patient comprising the step of administering to the patient in need of such treatment a therapeutically effective amount of 1-isopropyl-1′-(7-methoxy-2-naphthoyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering orally to the patient in need of such treatment a therapeutically effective amount of 1-isopropyl-1′-(7-methoxy-2-naphthoyl)-4,6-dihydrospiro[indazole-5,4′-piperidin]-7(1H)-one or pharmaceutically acceptable salt thereof.

In another embodiment, the present invention relates to a method of treating and/or preventing acne in a patient comprising the step of administering topically to the patient in need of such treatment a therapeutically effective amount of 1-isopropyl-1′-(7-methoxy-2-naphthoyl)-4,6-dihydrospiro[indazole-5,4′-piperidin]-7(1H)-one or pharmaceutically acceptable salt thereof.

The present invention relates to a method of treating and/or preventing acne in patients comprising the step of administering to patients in need of such treatment a pharmaceutical composition comprising a therapeutically effective amount of 1-isopropyl-1′-(7-methoxy-2-naphthoyl)-4,6-dihydrospiro[indazole-5,4′-piperidin]-7(1H)-one, or a pharmaceutically acceptable salt thereof, and at least one pharmaceutically acceptable carrier.

In another embodiment, the present invention relates to a method of reducing sebum triglycerides, sebum free fatty acids, cholesterol esters and sebum waxy esters in patients comprising the step of administering to patients in need of such treatment a therapeutically effective amount of 1-isopropyl-1′-(7-methoxy-2-naphthoyl)-4,6-dihydrospiro[indazole-5,4′-piperidin]-7(1H)-one or a pharmaceutically acceptable salt thereof.

In another embodiment, the present invention relates to a pharmaceutical combination comprising two different ACC inhibitors.

In another embodiment, the present invention relates to a pharmaceutical combination comprising an ACC inhibitor and an antibiotic, in particular, an antibiotic against P. acnes such as doxycycline, minocycline, tetracycline and erythromycin.

In another embodiment, the present invention relates to a pharmaceutical combination comprising an ACC inhibitor and one or more antibiotics, in particular, an antibiotic against P. acnes such as doxycycline, minocycline, tetracycline and/or erythromycin.

In another embodiment, the present invention relates to a pharmaceutical combination comprising an ACC inhibitor and an oral contraceptive.

In another embodiment, the present invention relates to a pharmaceutical combination comprising an ACC inhibitor and an androgen receptor blocker.

In another embodiment, the present invention relates to a pharmaceutical combination comprising an ACC inhibitor and a retinoid.

In another embodiment, the present invention relates to a pharmaceutical combination comprising an ACC inhibitor and benzoyl peroxide.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and a different ACC inhibitor.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and an antibiotic, in particular, an antibiotic against P. acnes such as doxycycline, minocycline, tetracycline and erythromycin.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and an oral contraceptive.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and an androgen receptor blocker.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and a retinoid.

In another embodiment, the present invention relates to a pharmaceutical combination comprising 1-isopropyl-1′-(2-methyl-1H-benzo[d]imidazole-5-carbonyl)-4,6-dihydrospiro[indazole-5,4′-piperidin]-7(1H)-one, or pharmaceutically acceptable salt thereof, and benzoyl peroxide.

The present invention includes the use of ACC inhibitors disclosed in the following patents and published patent applications WO03072197, WO13098375, WO13098373, WO13092976, US201358004, WO13079668, WO13071169, WO13061962, WO13035827, WO13017600, WO12108478, WO12104428, WO12090219, WO12074126, US2012129833, WO12056372, WO12032014, WO12028676, WO12013716, WO12001107, US2011263562, WO11129399, DE102010008642, DE102010008643, WO11098433, WO11067306, WO10127208, WO10127212, WO10050445, US2010113473, JP2010043019, WO10002010, US2009253725, JP2009196966, WO09055682, WO09023059, WO08140828, WO08121592, WO08102749, JP2008179621, WO08090944, WO08079610, WO08072850, WO08069500, WO07119833, WO07095603, WO07095601, WO07095602, WO07013691, WO06110775, US2006178400, WO06017494, US2008026363, WO03059886, WO03057255, U.S. Pat. No. 6,485,941, WO0134202, JP11171847 and JP11171848, each herein incorporated by reference, for treating acne in a patient via oral and/or topical administration.

### DEFINITIONS

The term “ACC inhibitor” as used herein means a compound that inhibits both ACC1 and ACC2. The ACC1/ACC2 assays disclosed herein may be used to establish inhibition activity (IC₅₀) for compounds against ACC1 and ACC2. A compound with an IC₅₀ below about 10 μM in the ACC1 and ACC2 assay is considered an ACC inhibitor. A preferred IC₅₀ is less than about 1 μM in both assays, and an especially preferred IC₅₀ is less than about 0.1 μM in both assays. In addition, ACC inhibitors of the present invention selectively inhibit ACC1 and ACC2 as compared to other enzymes, g-protein coupled receptors or ion channels. The compounds contemplated by the present invention inhibit other enzymes or bind (Ki) to receptors or ion channels at concentrations greater than the concentration required to inhibit ACC1 and ACC2. Preferred ACC inhibitory activity is about 2 to 10 fold greater than the IC₅₀ or Ki for other enzymes, receptors or ion channels, 10-100 fold is more preferred, and greater than 100 fold is especially preferred.

The term “Example 1” as used herein, means 1-isopropyl-1′-(2-methyl-1H-benzo[d]imidazole-5-carbonyl)-4,6-dihydrospiro[indazole-5,4′-piperidin]-7(1H)-one and includes the tautomer 1-isopropyl-1′-(2-methyl-1H-benzo[d]imidazole-6-carbonyl)-4,6-dihydrospiro[indazole-5,4′-piperidin]-7(1H)-one or combinations thereof. Example 1 may be prepared in a similar manner as described in U.S. Pat. No. 8,288,405.

The term “patient” as used herein, means a human.

The term “pharmaceutically acceptable salt” as used herein means those salts which are, within the scope of sound medical judgement, suitable for use in contact with the tissues of patients and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in Berge et al., J. Pharmaceutical Sciences, 1977, 66: 1-19. The salts can be prepared in situ during the final isolation and purification of Example 1 of the present invention or separately by reacting the free base of Example 1 with a suitable organic or inorganic acid. Representative acid addition salts include, but are not limited to acetate, adipate, alginate, citrate, aspartate, benzoate, benzenesulfonate, bicarbonate, bisulfate, butyrate, camphorate, camphorsufonate, citrate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, phosphate, picrate, pivalate, propionate, succinate, sulphate, tartrate, thiocyanate, and p-toluenesulfonate.

The present invention also provides pharmaceutical compositions which comprise an ACC inhibitor formulated together with one or more non-toxic pharmaceutically acceptable carriers. The pharmaceutical compositions may be specially formulated for oral administration in solid or liquid form, or for topical application.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise an ACC inhibitor formulated together with one or more non-toxic pharmaceutically acceptable carriers. The pharmaceutical compositions of this invention can be administered to patients orally or topically (as by powders, ointments or drops).

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

If desired, and for more effective distribution, an ACC inhibitor can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

An ACC inhibitor can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms an ACC inhibitor can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Solid dosage forms for oral administration include capsules, tablets, pills, powders, and granules. In such solid dosage forms an ACC inhibitor is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Liquid dosage forms for oral administration include pharmaceutically acceptable emulsions, microemulsions, solutions, suspensions, syrups and elixirs. In addition to an ACC inhibitor, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Actual dosage levels of an ACC inhibitor in the pharmaceutical compositions of this invention can be varied so as to obtain an amount of the ACC inhibitor which is effective to achieve the desired therapeutic response for a particular patient, compositions, and mode of administration. The selected dosage level will depend upon the activity of the ACC inhibitor, the route of administration, the severity of the condition being treated, and the condition and prior medical history of the patient being treated.

The total daily dose of an ACC inhibitor, in particular Example 1, administered to a patient is 0.3 to 800 mgs. A more preferred dosing range for Example 1 is 30 mg QD to 200 mg BID. If desired, the effective daily dose can be divided into multiple doses for purposes of administration, e.g. two to four separate doses per day.

### Example 1

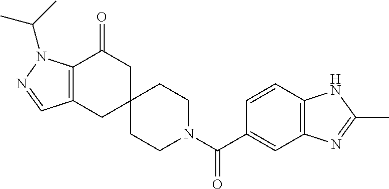 [SMILES: Cc1nc2cc(C(=O)N3CCC4(CC3)CC(=O)c3c(cnn3C(C)C)C4)ccc2[nH]1]

### 1-isopropyl-1′-(2-methyl-1H-benzo[d]imidazole-5-carbonyl)-4,6-dihydrospiro[indazole-5,4′-piperidin]-7(1H)-one

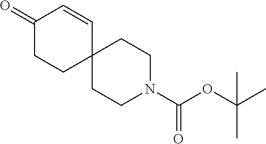 [SMILES: CC(C)(C)OC(=O)N1CCC2(C=CC(=O)CC2)CC1]

### tert-butyl 9-oxo-3-azaspiro[5.5]undec-7-ene-3-carboxylate

Methyl vinyl ketone (146 mL) was added to a solution of tert-butyl 4-formylpiperidine-1-carboxylate (375 g) in tetrahydrofuran (18 L). The reaction mixture was cooled to −5° C. and a solution of potassium hydroxide in ethanol (3N, 0.243 L) was added dropwise over 10 minutes. The reaction mixture was allowed to warm to room temperature and stirred for 16 hours. Cyclohexane (10 L) was added and the solution was washed with saturated sodium chloride (3×10 L). The organic layer was concentrated to an oil. This oil was dissolved in 2 L of 80:20 cyclohexane/ethyl acetate and filtered through Celite® to remove insoluble material. The filtrate was purified via flash column chromatography (70:30 hexane/ethyl acetate) to afford the product as an oil. The oil was triturated in hexanes to afford the desired product as a white solid (131 g, 28%).

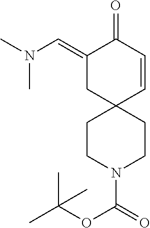 [SMILES: CN(C)/C=C1\CC2(C=CC1=O)CCN(C(=O)OC(C)(C)C)CC2]

### (E)-tert-butyl 10-((dimethylamino)methylene)-9-oxo-3-azaspiro[5.5]undec-7-ene-3-carboxylate

tert-Butyl 9-oxo-3-azaspiro[5.5]undec-7-ene-3-carboxylate (101 g), and tris(dimethylaminomethane) (133 mL) were dissolved in toluene (800 mL) and heated to reflux for 17 hours. The reaction mixture was concentrated to a minimum stirring volume and ethyl acetate (600 mL) was added. This mixture was heated to reflux and heptane (1.2 L) was added over 20 minutes. The hot solution was cooled to room temperature over 3 hours. The solids were filtered through a coarse glass frit and washed with heptane (300 mL). The resulting solid was dried in a vacuum oven at 40° C. for 3 hours to afford the desired product as a yellow solid (107 g). ¹H NMR (400 MHz, CDCl₃) δ ppm 7.48 (s, 1H), 6.57 (d, J=9.97 Hz, 1H), 5.99 (d, J=10.16 Hz, 1H), 3.32-3.51 (m, 4H), 3.06 (s, 6H), 2.72 (s, 2H), 1.57-1.66 (m, 2H), 1.41-1.53 (m, 11H).

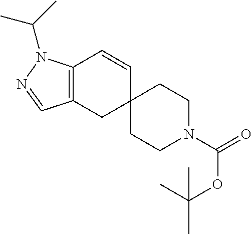 [SMILES: CC(C)n1ncc2c1C=CC1(CCN(C(=O)OC(C)(C)C)CC1)C2]

### tert-butyl 1-isopropyl-1,4-dihydrospiro[indazole-5,4′-piperidine]-1′-carboxylate

(E)-tert-butyl 10-((dimethylamino)methylene)-9-oxo-3-azaspiro[5.5]undec-7-ene-3-carboxylate (107 g) was taken up in toluene (700 mL) and isopropyl hydrazine (44.4 g) was added. The reaction was stirred at reflux for 4 hours. The reaction was cooled to room temperature and ethyl acetate was added (500 mL). The reaction solution was washed with citric acid (2×300 mL, 10% aqueous), and water (400 mL). The organic layer concentrated in vacuo to afford I-1A-1c as a yellow solid (109 g). ¹H NMR (400 MHz, CDCl₃) δ ppm 7.25 (s, 1H) 6.42 (dd, J=10.05, 0.49 Hz, 1H) 5.84 (d, J=9.95 Hz, 1H) 4.42-4.52 (m, 1H) 3.36-3.53 (m, 4H) 2.62 (s, 2H) 1.56-1.68 (m, 2H) 1.45-1.55 (m, 17H).

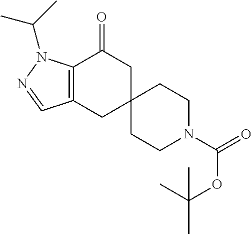 [SMILES: CC(C)n1ncc2c1C(=O)CC1(CCN(C(=O)OC(C)(C)C)CC1)C2]

### tert-butyl 1-isopropyl-7-oxo-1,4,6,7-tetrahydrospiro[indazole-5,4′-piperidine]-1′-carboxylate

To a solution of tert-butyl 1-isopropyl-1,4-dihydrospiro[indazole-5,4′-piperidine]-1′-carboxylate (109 g) in methanol (1 L) was added N-bromo succinimide (61.4 g). The reaction was stirred for 1 hour. The reaction was quenched with sodium thiosulfate (10 g in 300 mL water) and then distilled to a final volume of 500 mL. The solution was cooled to ambient temperature and 2-methyl tetrahydrofuran (1 L) and water (100 mL) were added. The organic layer was removed and the aqueous layer was extracted with 2-methyl tetrahydrofuran. The organic layers were combined, washed with aqueous sodium hydroxide (1 N, 250 mL), water, and saturated aqueous sodium chloride. The organic layer was dried over sodium sulfate, filtered and concentrated to an orange oil. The oil was dissolved in tetrahydrofuran (500 mL) and potassium tert-butoxide (76.8 g) in tetrahydrofuran (500 mL) was added. The solution was heated to 60° C. and stirred for 1 hour. Aqueous hydrochloric acid (1 N, 1 L) was added and the solution was stirred for 30 minutes. The phases were separated and the aqueous layer was extracted with ethyl acetate (700 mL). The organic layers were combined, washed with water (400 mL) and saturated aqueous sodium chloride (100 mL). The organic layer was dried over sodium sulfate, filtered and concentrated in vacuo to give a residue. The residue was dried in a vacuum oven at 40° C. for 16 hours to afford the title compound as an orange wax (117 g). ¹H NMR (400 MHz, CDCl₃) δ ppm 7.35 (s, 1H), 5.32-5.42 (m, 1H), 3.29-3.51 (m, 4H), 2.73 (s, 2H), 2.51 (s, 2H), 1.47-1.57 (m, 4H), 1.42-1.46 (m, 15H); +ESI MS (M+H)=348.5.

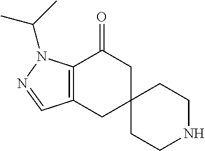 [SMILES: CC(C)n1ncc2c1C(=O)CC1(CCNCC1)C2]

### 1-isopropyl-4,6-dihydrospiro[indazole-5,4′-piperidin]-7(1H)-one

tert-Butyl 1-isopropyl-7-oxo-1,4,6,7-tetrahydrospiro[indazole-5,4′-piperidine]-1′-carboxylate (23.5 g) was suspended in ethyl acetate (260 mL) and methanol (70 mL). The reaction solution was cooled to <2° C. and acetyl chloride (33.6 mL) was added dropwise over 20 min. The reaction was allowed to slowly warm to room temperature and was stirred for 4 hours. The reaction solution was cooled to 0° C. and the precipitate was filtered. The precipitate was washed with ethyl acetate (200 mL) and dried in a vacuum oven (40° C., 10 mm Hg) for 16 hours to afford the title compound as a light yellow solid. ¹H NMR (400 MHz, CD₃OD) δ ppm 7.43 (s, 1H), 5.32-5.42 (m, 1H), 3.15-3.25 (m, 4H), 2.89 (s, 2H), 2.64 (s, 2H), 1.69-1.90 (m, 4H), 1.37-1.45 (m, 6H); +ESI MS (M+H)=248.4.

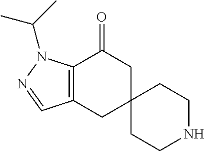 [SMILES: CC(C)n1ncc2c1C(=O)CC1(CCNCC1)C2]

### Alternate Preparation of

### 1-isopropyl-4,6-dihydrospiro[indazole-5,4′-piperidin]-7(1H)-one Preparation

tert-Butyl 9-oxo-3-azaspiro[5.5]undec-7-ene-3-carboxylate (250 g), and tris (dimethylaminomethane) (325 mL) were dissolved in toluene (1.9 L) and heated at reflux for 4 hours. The mixture was distilled and concentrated to a minimum stirring volume (110° C.) and then toluene (1.9 L) was added. The reaction was redistilled to a minimum stirring volume and cooled to room temperature. Toluene (1.8 L) and isopropyl hydrazine hydrochloride (135 g) were added and the solution was heated to reflux for 5 hours. The reaction was cooled to room temperature and was then washed with citric acid (10% aqueous, 2×150 mL) and water (200 mL), and then the organic layer was distilled to a minimum stirring volume. Methanol (2 L) was added and distilled to a minimum stirring volume. This was repeated with methanol (2 L). The solution was redissolved in methanol (2.5 L) and N-bromosuccinimide (176 g) was added in one portion. The solution was stirred at 23° C. for 2 hours. Aqueous sodium thiosulfate solution (5 wt %, 0.5 L) was added and the mixture was stirred for 15 minutes. The reaction mixture was concentrated via distillation (45° C., 210 mm Hg) to ˜0.5 L and then 2-methyl tetrahydrofuran (2.5 L) was added. After stirring for 15 minutes the aqueous layer was discarded. The organic layer was concentrated to ˜0.2 L and tetrahydrofuran (0.5 L) was added. To the mixture was added a potassium tert-butoxide solution in tetrahydrofuran (1.9 L, 1 M solution). The solution was heated to 60° C. and stirred for 1 hour. After cooling to room temperature, aqueous hydrochloric acid (1 N, 2.2 L) was added over 20 minutes. The mixture was stirred at room temperature for 20 minutes, and then the layers were allowed to separate. The aqueous layer was removed and back extracted with ethyl acetate (1.75 L). The combined organic layers were washed with water (1 L) and the organic layer concentrated via distillation (4 L solvent removed). Ethyl acetate (1.8 L) was added and the solution was concentrated to a minimum stirring volume. Ethyl acetate (3 L) and methanol (0.8 L) were added and the solution was cooled to 0° C. Acetyl chloride (401 mL) was added dropwise over 20 minutes and the solution was stirred at 0° C. for 4 hours. The precipitate was collected by filtration under nitrogen. The filtrate was washed with ethyl acetate (0.5 L) and dried in a vacuum oven at 40° C. to afford the I-1A-1e as an off-white solid (241 g). ¹H NMR (400 MHz, CD₃OD) δ ppm 7.43 (s, 1H), 5.32-5.42 (m, 1H), 3.15-3.25 (m, 4H), 2.89 (s, 2H), 2.64 (s, 2H), 1.69-1.90 (m, 4H), 1.37-1.45 (m, 6H); +ESI (M+H)=248.4

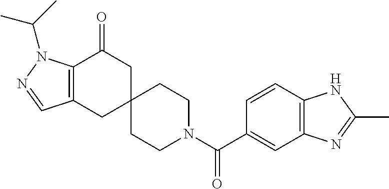 [SMILES: Cc1nc2cc(C(=O)N3CCC4(CC3)CC(=O)c3c(cnn3C(C)C)C4)ccc2[nH]1]

### 1-isopropyl-1′-(2-methyl-1H-benzo[d]imidazole-5-carbonyl)-4,6-dihydrospiro[indazole-5,4′-piperidin]-7(1H)-one

2-Methyl-1H-benzimidazole-5-carboxylic acid (15 g) was taken up in tetrahydrofuran (500 mL), dimethylformamide (329 uL) and oxalyl chloride (22.1 mL) were added. The reaction solution was stirred at ambient temperature for 16 hours. The solution was concentrated in vacuo and the residue was taken up in dichloromethane and concentrated (×2) under reduced pressure. To the resulting acid chloride was added tetrahydrofuran (500 mL), 1-isopropyl-4,6-dihydrospiro[indazole-5,4′-piperidin]-7(1H)-one (25.9 g) and triethylamine (71.2 mL). The solution was stirred at room temperature for 16 hours. To the reaction was added saturated, aqueous sodium bicarbonate (250 mL) and the solution was stirred for 5 min. The layers were separated and the aqueous layer was extracted with 1:1 ethyl acetate/tetrahydrofuran. The organic layers were combined, diluted with ethyl acetate (1 L) and washed with saturated aqueous, sodium bicarbonate (200 mL) and saturated, aqueous sodium chloride. The organic layer was dried over sodium sulfate, filtered and concentrated in vacuo to a light yellow solid. The solid was dissolved in hot methanol (300 mL) and then heated to reflux. To the solution was added 350 mL ethyl acetate and 300 mL of solvent was removed by distillation. Additional ethyl acetate was added dropwise until an internal temperature of 70° C. was reached. The solution was cooled to room temperature over 3 hours. The solids were collected by filtration and dried in a vacuum oven (40° C.) for 16 hours to afford the title compound as a white solid (20.5 g, 59%): +ESI MS (M+H) 406.5; ¹H NMR (400 MHz, DMSO-d₆) δ ppm 12.25-12.33 (m, 1H), 7.35-7.51 (m, 3H), 7.05-7.16 (m, 1H), 5.16-5.31 (m, 1H), 3.32-3.58 (m, 4H), 2.77 (s, 2H), 2.57 (s, 2H), 1.40-1.52 (m, 4H), 1.32 (d, J=6.45 Hz, 6H).

In the present example it is to be understood that the starting material 2-Methyl-1H-benzimidazole-5-carboxylic acid employed in this example also exists as its tautomeric form 2-Methyl-1H-benzimidazole-6-carboxylic acid (also known as 2-Methyl-3H-benzimidazole-5-carboxylic acid) and each is designated by the same CAS No. 709-19-3. It is to be further understood that the instant example has been depicted above as one of two tautomeric forms of the compound with respect to the 2-methyl benzimidazolyl group and that 1-isopropyl-1′-(2-methyl-1H-benzo[d]imidazole-5-carbonyl)-4,6-dihydrospiro[indazole-5,4′-piperidin]-7(1H)-one is synonomous with the tautomeric form 1-isopropyl-1′-(2-methyl-1H-benzo[d]imidazole-6-carbonyl)-4,6-dihydrospiro[indazole-5,4′-piperidin]-7(1H)-one which is depicted as:

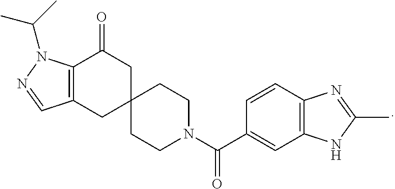 [SMILES: Cc1nc2ccc(C(=O)N3CCC4(CC3)CC(=O)c3c(cnn3C(C)C)C4)cc2[nH]1]

### Example 2

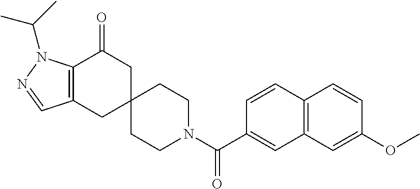 [SMILES: COc1ccc2ccc(C(=O)N3CCC4(CC3)CC(=O)c3c(cnn3C(C)C)C4)cc2c1]

### 1-isopropyl-1′-(7-methoxy-2-naphthoyl)-4,6-dihydrospiro[indazole-5,4′-piperidin]-7(1H)-one

To a solution of 7-methoxy-2-naphthoic acid (202 mg, 1.00 mmol) and 1-isopropyl-4,6-dihydrospiro[indazole-5,4′-piperidin]-7(1H)-one (329 mg, 1.05 mmol) in dichloromethane (15 mL) was added triethylamine (304 mg, 3.00 mmol) and then 1-hydroxybenzotriazole (149 mg, 1.10 mmol). The reaction mixture was stirred at room temperature for 15 minutes and then 1-ethyl-3-(3-dimethylaminopropyl)carbodiimide was added (211 mg, 1.10 mmol) and the reaction was stirred for 15 hours. The mixture was then diluted with dichloromethane and washed with saturated aqueous sodium bicarbonate and brine. The organic phase was dried over magnesium sulfate, filtered and concentrated under reduced pressure. The resultant residue was purified by flash chromatography (20-100% 1:9 methanol in ethyl acetate/heptane, 24 g RediSep® Gold column) to yield 342 mg (79%) of 1-isopropyl-1′-(7-methoxy-2-naphthoyl)-4,6-dihydrospiro[indazole-5,4′-piperidin]-7(1H)-one as a colorless foam. +ESI (M+H) 432.3; ¹H NMR (600 MHz, CHLOROFORM-d) δ ppm 1.38-1.50 (m, 6H) 1.53 (br. s., 1H) 1.60 (br. s., 1H) 1.71 (br. s., 2H) 2.60 (s, 2H) 2.81 (s, 2H) 3.47 (d, J=14.7 Hz, 2H) 3.79 (d, J=14.1 Hz, 1H) 3.86 (br. s., 1H) 3.92 (s, 3H) 5.37 (dt, J=13.4, 6.5 Hz, 1H) 7.13 (d, J=2.4 Hz, 1H) 7.19 (dd, J=9.4, 2.4 Hz, 1H) 7.31 (d, J=9.4 Hz, 1H) 7.38 (s, 1H) 7.74 (d, J=8.8 Hz, 1H) 7.76-7.79 (m, 2H)

### Example 3

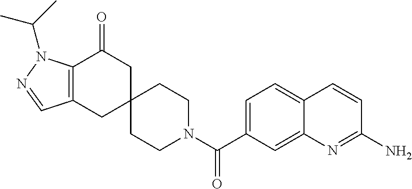 [SMILES: CC(C)n1ncc2c1C(=O)CC1(CCN(C(=O)c3ccc4ccc(N)nc4c3)CC1)C2]

### 1′-(2-aminoquinoline-7-carbonyl)-1-isopropyl-4,6-dihydrospiro[indazole-5,4′-piperidin]-7(1H)-one

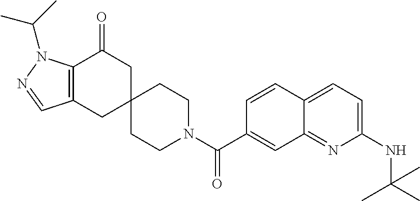 [SMILES: CC(C)n1ncc2c1C(=O)CC1(CCN(C(=O)c3ccc4ccc(NC(C)(C)C)nc4c3)CC1)C2]

### 1′-(2-(tert-butylamino)quinoline-7-carbonyl)-1-isopropyl-4,6-dihydrospiro[indazole-5,4′-piperidin]-7(1H)-one

The title compound was prepared by a method analogous to that described in Example 25, step 1 of US 2012/0270893 herein incorporated by reference. +APCI (M+H) 474.6; ¹H NMR (400 MHz, CDCl₃, δ): 7.72 (d, J=8.8 Hz, 1H), 7.64 (s, 1H), 7.55 (d, J=8.2 Hz, 1H), 7.36 (s, 1H), 7.16 (dd, J=8.1, 1.3 Hz, 1H), 6.59 (d, J=9.2 Hz, 1H), 5.36 (quin, J=6.6 Hz, 1H), 3.31-3.96 (m, 4H), 2.79 (s, 2H), 2.58 (s, 2H), 1.55-1.75 (m, 4H), 1.52 (s, 9H), 1.44 (d, J=6.4 Hz, 6H).

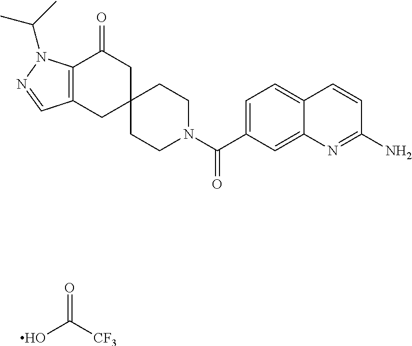

### 1′-[(2-aminoquinolin-7-yl)carbonyl]-1-isopropyl-1,4-dihydrospiro[indazole-5,4′-piperidin]-7(6H)-one Trifluoroacetate Salt

Trifluoroacetic acid (0.90 mL, 12 mmol) was added to 1′-(2-(tert-butylamino)quinoline-7-carbonyl)-1-isopropyl-4,6-dihydrospiro[indazole-5,4′-piperidin]-7(1H)-one (50 mg, 0.11 mmol). The reaction was heated to 70° C. for 3 hours, then cooled to room temperature and left stirring overnight. The reaction was concentrated to dryness and purification by reversed-phase HPLC gave the title compound (41 mg, 93%). HPLC retention time 2.11 minutes measured using Waters Atlantis dC18 4.6×50 mm, 5 μm, column; mobile phase A: 0.05% TFA in water (v/v); mobile phase B: 0.05% TFA in acetonitrile (v/v); gradient: 95% A/5% B linear to 5% A/95% B in 4.0 minutes, hold at 5% A/95% B for 5.0 minutes; flow rate: 2.0 mL/minute. +ESI (M+H) 418.2; ¹H NMR (500 MHz, CD₃OD, δ) 8.36 (d, J=9.27 Hz, 1H), 7.97 (d, J=8.05 Hz, 1H), 7.66 (s, 1H), 7.53 (dd, J=8.17, 1.34 Hz, 1H), 7.44 (s, 1H), 7.12 (d, J=9.27 Hz, 1H), 5.39 (quint, J=13.23, 6.68 Hz, 1H), 3.91 (br. s., 1H), 3.76 (br. s., 1H), 3.46 (br. s., 2H), 2.92 (s, 2H), 2.67 (d, J=7.81 Hz, 2H), 1.74 (br. s., 2H), 1.59 (br. s., 2H), 1.43 (br. s., 6H).

### 1′-[(2-aminoquinolin-7-yl)carbonyl]-1-isopropyl-1,4-dihydrospiro[indazole-5,4′-piperidin]-7(6H)-one

### Example 4

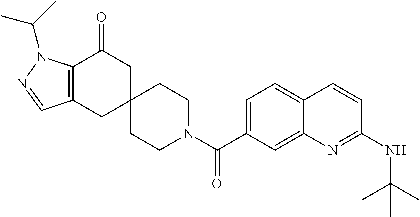 [SMILES: CC(C)n1ncc2c1C(=O)CC1(CCN(C(=O)c3ccc4ccc(NC(C)(C)C)nc4c3)CC1)C2]

### 1′-(2-(tert-butylamino)quinoline-7-carbonyl)-1-isopropyl-4,6-dihydrospiro[indazole-5,4′-piperidin]-7(1H)-one

The title compound was prepared by a method analogous to that described in Example 25, step 1 of US 2012/0270893 herein incorporated by reference. +APCI (M+H) 474.6; ¹H NMR (400 MHz, CDCl₃, δ): 7.72 (d, J=8.8 Hz, 1H), 7.64 (s, 1H), 7.55 (d, J=8.2 Hz, 1H), 7.36 (s, 1H), 7.16 (dd, J=8.1, 1.3 Hz, 1H), 6.59 (d, J=9.2 Hz, 1H), 5.36 (quin, J=6.6 Hz, 1H), 3.31-3.96 (m, 4H), 2.79 (s, 2H), 2.58 (s, 2H), 1.55-1.75 (m, 4H), 1.52 (s, 9H), 1.44 (d, J=6.4 Hz, 6H).

### Example 5

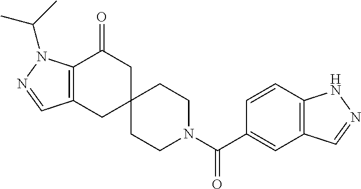 [SMILES: CC(C)n1ncc2c1C(=O)CC1(CCN(C(=O)c3ccc4[nH]ncc4c3)CC1)C2]

### 1′-(1H-indazole-5-carbonyl)-1-isopropyl-4,6-dihydrospiro[indazole-5,4′-piperidin]-7(1H)-one

May be prepared as described in Example 10A-1 of US 2011/0111046 herein incorporated by reference.

### Example 6

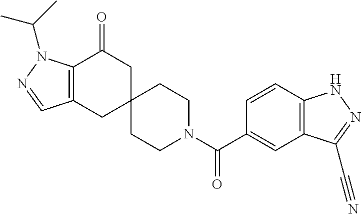 [SMILES: CC(C)n1ncc2c1C(=O)CC1(CCN(C(=O)c3ccc4[nH]nc(C#N)c4c3)CC1)C2]

### 5-(1-isopropyl-7-oxo-1,4,6,7-tetrahydrospiro[indazole-5,4′-piperidin]-1′-ylcarbonyl)-1H-indazole-3-carbonitrile

May be prepared as described in Example 11A-9 of US 2011/0111046 herein incorporated by reference.

### Example 7

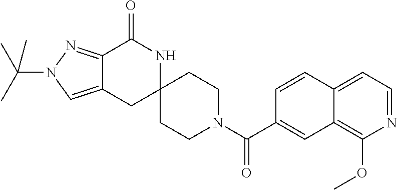 [SMILES: COc1nccc2ccc(C(=O)N3CCC4(CC3)Cc3cn(C(C)(C)C)nc3C(=O)N4)cc12]

### 2′-(tert-butyl)-1-(1-methoxyisoquinoline-7-carbonyl)-4′,6′-dihydrospiro[piperidine-4,5′-pyrazolo[3,4-c]pyridin]-7′(2′H)-one

May be prepared as described in Example 55 of WO 2012/056372 herein incorporated by reference.

### Example 8

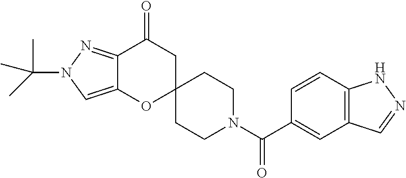 [SMILES: CC(C)(C)n1cc2c(n1)C(=O)CC1(CCN(C(=O)c3ccc4[nH]ncc4c3)CC1)O2]

### 2′-(tert-butyl)-1-(1H-indazole-5-carbonyl)-2′H-spiro[piperidine-4,5′-pyrano[3,2-c]pyrazol]-7′(6′H)-one

May be prepared as described in Example 1.074 of WO 2009/144554 herein incorporated by reference.

### Example 9

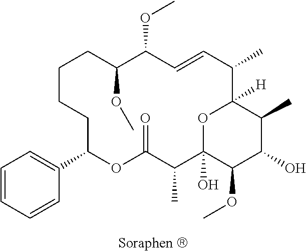 [SMILES: CO[C@H]1CCCC[C@@H](c2ccccc2)OC(=O)[C@@H](C)[C@@]2(O)O[C@H]([C@@H](C)[C@H](O)[C@H]2OC)[C@@H](C)/C=C/[C@H]1OC]

May be purchased commercially.

### Example 10

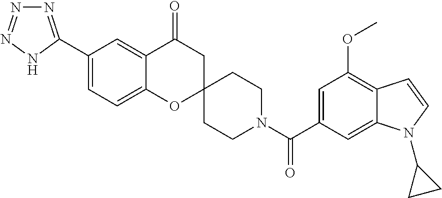 [SMILES: COc1cc(C(=O)N2CCC3(CC2)CC(=O)c2cc(-c4nnn[nH]4)ccc2O3)cc2c1ccn2C1CC1]

### 1′-(1-cyclopropyl-4-methoxy-1H-indole-6-carbonyl)-6-(1H-tetrazol-5-yl)spiro[chromane-2,4′-piperidin]-4-one

May be prepared as described in US 2008/0171761 herein incorporated by reference.

### Example 11

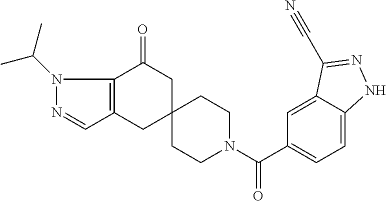 [SMILES: CC(C)n1ncc2c1C(=O)CC1(CCN(C(=O)c3ccc4[nH]nc(C#N)c4c3)CC1)C2]

### 5-(1-isopropyl-7-oxo-1,4,6,7-tetrahydrospiro[indazole-5,4′-piperidine]-1′-carbonyl)-1H-indazole-3-carbonitrile

May be prepared as described in US 2011/0111046 herein incorporated by reference.

### Example 12

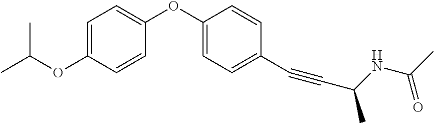 [SMILES: CC(=O)N[C@@H](C)C#Cc1ccc(Oc2ccc(OC(C)C)cc2)cc1]

### (S)—N-(4-(4-(4-isopropoxyphenoxy)phenyl)but-3-yn-2-yl)acetamide

May be prepared as described in US 2006/0178400 herein incorporated by reference.

### Example 13

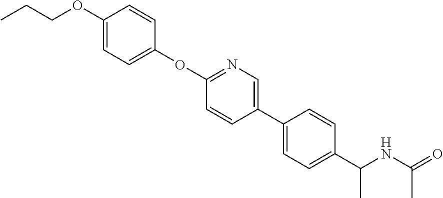 [SMILES: CCCOc1ccc(Oc2ccc(-c3ccc(C(C)NC(C)=O)cc3)cn2)cc1]

### N-(1-(4-(6-(4-propoxyphenoxy)pyridin-3-yl)phenyl)ethyl)acetamide

May be prepared as described in BMCL 2009, 5872, cmpd 11a/11b herein incorporated by reference.

### Example 14

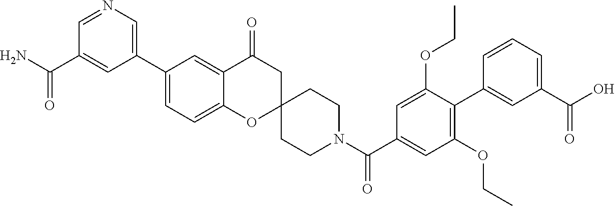 [SMILES: CCOc1cc(C(=O)N2CCC3(CC2)CC(=O)c2cc(-c4cncc(C(N)=O)c4)ccc2O3)cc(OCC)c1-c1cccc(C(=O)O)c1]

### 4′-(6-(5-carbamoylpyridin-3-yl)-4-oxospiro[chromane-2,4′-piperidine]-1′-carbonyl)-2′,6′-diethoxy-[1,1′-biphenyl]-3-carboxylic acid

May be prepared as described in WO 2010/002010 herein incorporated by reference.

### Example 15

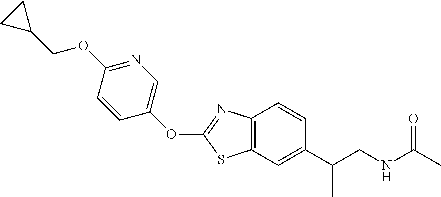 [SMILES: CC(=O)NCC(C)c1ccc2nc(Oc3ccc(OCC4CC4)nc3)sc2c1]

### N-(2-(2-((6-(cyclopropylmethoxy)pyridin-3-yl)oxy)benzo[d]thiazol-6-yl)propyl)acetamide

May be prepared as described in WO 2007/095601 herein incorporated by reference.

### Example 16

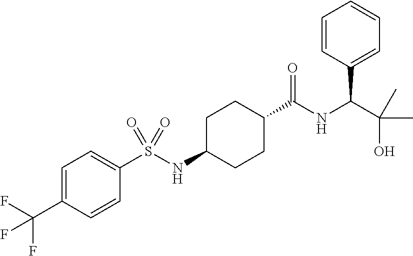 [SMILES: CC(C)(O)[C@@H](NC(=O)[C@H]1CC[C@H](NS(=O)(=O)c2ccc(C(F)(F)F)cc2)CC1)c1ccccc1]

### (1R,4S)—N—((S)-2-hydroxy-2-methyl-1-phenylpropyl)-4-((4-(trifluoromethyl)phenyl)sulfonamido)cyclohexane-1-carboxamide

May be prepared as described in WO 2011/0637306 herein incorporated by reference.

### Example 17

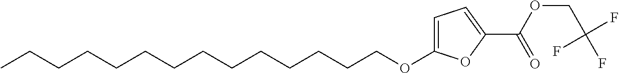 [SMILES: CCCCCCCCCCCCCCOc1ccc(C(=O)OCC(F)(F)F)o1]

### 2,2,2-trifluoroethyl 5-(tetradecyloxy)furan-2-carboxylate

May be prepared as described in US 2012/0208807.

### Example 18

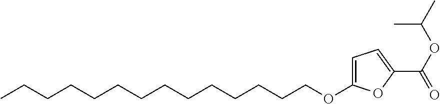 [SMILES: CCCCCCCCCCCCCCOc1ccc(C(=O)OC(C)C)o1]

### isopropyl 5-(tetradecyloxy)furan-2-carboxylate

May be prepared as described in US 2012/0208807.

### Example 19

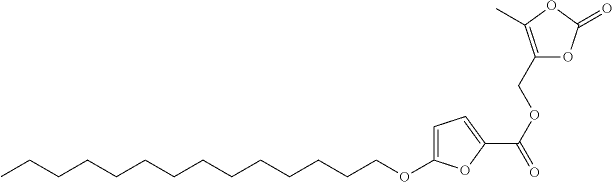 [SMILES: CCCCCCCCCCCCCCOc1ccc(C(=O)OCc2oc(=O)oc2C)o1]

### (5-methyl-2-oxo-1,3-dioxol-4-yl)methyl 5-(tetradecyloxy)furan-2-carboxylate

May be prepared as described in US 2012/0208807.

### Example 20

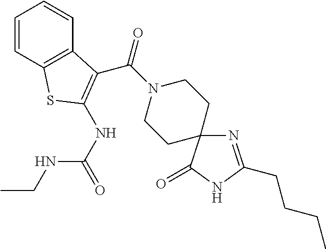 [SMILES: CCCCC1=NC2(CCN(C(=O)c3c(NC(=O)NCC)sc4ccccc34)CC2)C(=O)N1]

### 1-(3-(2-butyl-4-oxo-1,3,8-triazaspiro[4.5]dec-1-ene-8-carbonyl)benzo[b]thiophen-2-yl)-3-ethyl urea

May be prepared as described in US 2009/0253725 herein incorporated by reference.

### Example 21

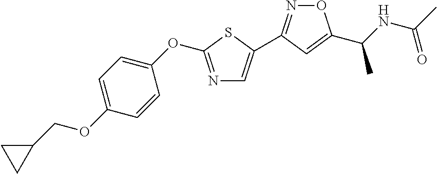 [SMILES: CC(=O)N[C@@H](C)c1cc(-c2cnc(Oc3ccc(OCC4CC4)cc3)s2)no1]

### (S)—N-(1-(3-(2-(4-(cyclopropylmethoxy)phenoxy)thiazol-5-yl)isoxazol-5-yl)ethyl)acetamide

May be prepared as described in WO 2007/095602 herein incorporated by reference.

### Example 22

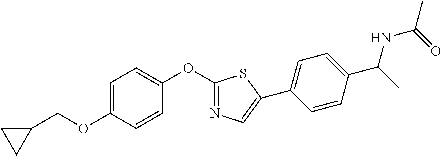 [SMILES: CC(=O)NC(C)c1ccc(-c2cnc(Oc3ccc(OCC4CC4)cc3)s2)cc1]

### N-(1-(4-(2-(4-(cyclopropylmethoxy)phenoxy)thiazol-5-yl)phenyl)ethyl)acetamide

May be prepared as described in WO 2007/095603 herein incorporated by reference.

### Example 23

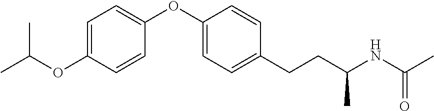 [SMILES: CC(=O)N[C@@H](C)CCc1ccc(Oc2ccc(OC(C)C)cc2)cc1]

### (S)—N-(4-(4-(4-isopropoxyphenoxy)phenyl)butan-2-yl)acetamide

May be prepared as described in WO 2008/079610 herein incorporated by reference.

### Example 24

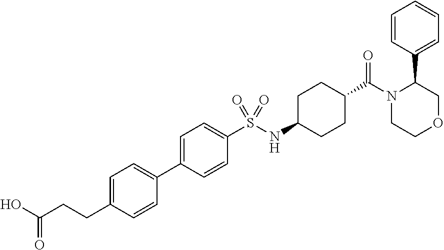 [SMILES: O=C(O)CCc1ccc(-c2ccc(S(=O)(=O)N[C@H]3CC[C@H](C(=O)N4CCOC[C@@H]4c4ccccc4)CC3)cc2)cc1]

### 3-(4′-(N-((1S,4r)-4-((S)-3-phenylmorpholine-4-carbonyl)cyclohexyl)sulfamoyl)-[1,1′-biphenyl]-4-yl)propanoic acid

May be prepared as described in WO 2011/0637306 herein incorporated by reference.

### Biological Protocols

The utility of the compounds of present invention in the treatment and/or prevention of acne vulgaris in patients may be demonstrated by the activity in the in vitro and in vivo assays described below. Such assays also provide a means whereby the activities of the compounds of the present invention can be compared with the activities of other known compounds.

### Direct Inhibition of the Activities of ACC1 and ACC2

The ACC inhibitory activity of the compounds of the present invention was demonstrated by methods based on standard procedures. The direct inhibition of ACC1 and ACC2 activity for the compounds of the present invention was determined using preparations of recombinant human ACC1 (rhACC1) (SEQ ID NO. 1) and recombinant human ACC2 (rhACC2) (SEQ ID NO. 2).

### Preparation of rhACC1

Two liters of SF9 cells, infected with recombinant baculovirus containing full length human ACC1 cDNA, were suspended in ice-cold lysis buffer (25 mM Tris, pH 7.5; 150 mM NaCl; 10% glycerol; 5 mM imidazole (EMD Bioscience; Gibbstown, N.J.); 2 mM TCEP (BioVectra; Charlottetown, Canada); Benzonase nuclease (10000 U/100 g cell paste; Novagen; Madison, Wis.); EDTA-free protease inhibitor cocktail (1 tab/50 mL; Roche Diagnostics; Mannheim, Germany). Cells were lysed by 3 cycles of freeze-thaw and centrifuged at 40,000×g for 40 minutes (4° C.). Supernatant was directly loaded onto a HisTrap FF crude column (GE Healthcare; Piscataway, N.J.) and eluted with an imidazole gradient up to 0.5 M over 20 column volumes (CV). ACC1-containing fractions were pooled and diluted 1:5 with 25 mM Tris, pH 7.5, 2 mM TCEP, 10% glycerol and direct loaded onto a CaptoQ (GE Healthcare) column and eluted with an NaCl gradient up to 1 M over 20 CV's. Phosphate groups were removed from purified ACC1 by incubation with lambda phosphatase (100 U/10 μM target protein; New England Biolabs; Beverly, Mass.) for 14 hours at 4° C.; okadaic acid was added (1 μM final concentration; Roche Diagnostics) to inhibit the phosphatase. Purified ACC1 was exchanged into 25 mM Tris, pH 7.5, 2 mM TCEP, 10% glycerol, 0.5 M NaCl by 6 hour dialysis at 4° C. Aliquots were prepared and frozen at −80° C.

Measurement of rhACC1 Inhibition.

hACC1 was assayed in a Costar #3676 (Costar, Cambridge, Mass.) 384-well plate using the Transcreener ADP detection FP assay kit (Bellbrook Labs, Madison, Wis.) using the manufacturer's recommended conditions for a 50 μM ATP reaction. The final conditions for the assay were 50 mM HEPES, pH 7.2, 10 mM MgCl₂, 7.5 mM tripotassium citrate, 2 mM DTT, 0.1 mg/mL BSA, 30 μM acetyl-CoA, 50 μM ATP, and 10 mM KHCO₃. Typically, a 10 μl reaction was run for 120 min at 25° C., and 10 μl of Transcreener stop and detect buffer was added and the combination incubated at room temp for an additional 1 hour. The data was acquired on a Envision Fluorescence reader (PerkinElmer) using a 620 excitation Cy5 FP general dual mirror, 620 excitation Cy5 FP filter, 688 emission (S) and a 688 (P) emission filter.

### Preparation of rhACC2

Human ACC2 inhibition was measured using purified recombinant human ACC2 (hrACC2). Briefly, a full length Cytomax clone of ACC2 was purchased from Cambridge Bioscience Limited and was sequenced and subcloned into PCDNA5 FRT TO-TOPO (Invitrogen, Carlsbad, Calif.). The ACC2 was expressed in CHO cells by tetracycline induction and harvested in 5 liters of DMEM/F12 with glutamine, biotin, hygromycin and blasticidin with 1 μg/mL tetracycline (Invitrogen, Carlsbad, Calif.). The conditioned medium containing ACC2 was then applied to a Softlink Soft Release Avidin column (Promega, Madison, Wis.) and eluted with 5 mM biotin. 4 mgs of ACC2 were eluted at a concentration of 0.05 mg/mL (determined by A280) with an estimated purity of 95% (determined by A280). The purified ACC2 was dialyzed in 50 mM Tris, 200 mM NaCl, 4 mM DTT, 2 mM EDTA, and 5% glycerol. The pooled protein was frozen and stored at −80° C., with no loss of activity upon thawing. For measurement of ACC2 activity and assessment of ACC2 inhibition, test compounds were dissolved in DMSO and added to the rhACC2 enzyme as a 5× stock with a final DMSO concentration of 1%.

### Measurement of Human ACC2 Inhibition

hACC2 was assayed in a Costar #3676 (Costar, Cambridge, Mass.) 384-well plate using the Transcreener ADP detection FP assay kit (Bellbrook Labs, Madison, Wis.) using the manufacturer's recommended conditions for a 50 uM ATP reaction. The final conditions for the assay were 50 mM HEPES, pH 7.2, 5 mM MgCl₂, 5 mM tripotassium citrate, 2 mM DTT, 0.1 mg/mL BSA, 30 μM acetyl-CoA, 50 μM ATP, and 8 mM KHCO₃. Typically, a 10 μl reaction was run for 50 min at 25° C., and 10 μl of Transcreener stop and detect buffer was added and the combination incubated at room temp for an additional 1 hour. The data was acquired on an Envision Fluorescence reader (PerkinElmer) using a 620 excitation Cy5 FP general dual mirror, 620 excitation Cy5 FP filter, 688 emission (S) and a 688 (P) emission filter.

The results using the recombinant hACC1 and recombinant hACC2 Transcreener assays described above are summarized in the table 1 below.

### Inhibition of De Novo Lipogenesis in Cultured Human Sebocytes

SZ95 sebocytes were grown in Human Sebocyte Growth Medium (HSGM) (Sebomed® medium (Biochrom: F8205) supplemented with 10% heat-inactivated fetal bovine serum, 1% penicillin/streptomycin, 1 mM calcium chloride and 5 ng/mL recombinant human epidermal growth factor (Life Tech: PHG0311)). At 90% confluence, cells were washed three times with PBS and then detached with 0.05% Trypsin-EDTA. Cells were centrifuged and resuspended in HSGM containing 10% charcoal-stripped serum (Life Tech: 12676-029). Cells were then plated in 24-well plates at a density of 0.25×10⁶ cells/well and were incubated overnight to enable cell adherence to the culture plate.

Cells were treated with a dose response of compound, and each dose was assayed in triplicate. Briefly, compounds were dissolved in DMSO stocks and diluted 1:1000 into charcoal-stripped media. 0.1% DMSO without compound was used as the vehicle control. After a 1 hour preincubation with compound or vehicle, 0.25 μCi ¹⁴C-acetic acid (American Radiolabeled Chemicals: ARC0158B) was added to each well. Plates were then incubated for an additional two hours. At the end of the incubation period, cells were removed from incubator, placed on ice, and then washed twice with ice-cold PBS to remove free ¹⁴C-acetic acid. Plates were sealed and stored at −20° C. until analysis.

125 μl mammalian protein extraction reagent (MPER, Pierce: 78505) was added to each well. Plates were shaken for 1 hour at room temperature to induce lysis. Lysates were transferred to individual 1.5 mL polypropylene tubes, and wells were washed with 175 μl of PBS, which was added to lysates. 450 μl of a 1:1 (v/v) chloroform:methanol solution was then added to each tube, and then all tubes were vortexed for 10 seconds and centrifuged at 20,000×g for 5 minutes at room temperature to separate aqueous from organic phase. A 25 μl aliquot was removed from the bottom organic layer of each sample and added to 6 mL OptiPhase supermix (PerkinElmer: 1200-439) scintillation fluid. Counts of ¹⁴C incorporated into lipids were assessed by scintillation counting. DNL (counts of ¹⁴C incorporated into lipids) was expressed for compound treated cells relative to DNL in the vehicle control treated cells for determination of EC₅₀ values. For a subset of the compounds tested, a second aliquot (3×35 μl) of the organic layer of each sample was removed and applied to a TLC lane (Analtech Silica Gel G Plates). Radiolabeled lipids were resolved using a 2-solvent system. Solvent 1 contained a 100:100:100:40:36 mixture of ethyl acetate:isopropyl alcohol:CHCl3:MeOH:0.25% KCl and solvent 2 a 70:27:3 hexane:diethyl ether:acetic acid mix. The TLC plate was dried under nitrogen for 30 minutes and [¹⁴C]-calibrators added to a vacant lane. Bands were visualized and quantitated using a Molecular Dynamics' Storm 860 Phosphorimager system following 18-36 hours exposure to a Phosphorimager screen.

The results for the inhibition of de novo lipogenesis in cultured human sebocytes described above are summarized in table 1 below and dose response curves (plotted as percent of vehicle control) are shown for Example 1 and Example 3 in FIG. 2. Visualization using TLC of the effect of Example 3 vs. vehicle on sebocycte lipid classes is shown in FIG. 4.

TABLE 1

| | Trans- | | Trans- | | | |
|---|---|---|---|---|---|---|
| | screener | | screener | | | |
| | Assay | | Assay | | C9302E | |
| Example | hACC1 IC₅₀ | | hACC2 IC₅₀ | | Sebocyte | |
| Number | (nM) | n¹ | (nM) | n¹ | IC₅₀ (nM) | n¹ |
| 1 | 98 | 12 | 45 | 21 | 679 | 6 |
| 2 | 4 | 5 | 2 | 5 | 28.2 | 3 |
| 3 | 12 | 2 | 10 | 2 | 90 | 5 |
| 4 | 17 | 1 | 12 | 1 | 15 | 3 |
| 5 | 62 | 8 | 30 | 9 | 201 | 3 |
| 6 | 6 | 4 | 3 | 4 | 103 | 1 |
| 7 | 14 | 4 | 3 | 4 | 94 | 4 |
| 8 | 111 | 7 | 10 | 27 | 549 | 8 |
| 9 | 20 | 4 | 1200 | 8 | 4 | 1 |
| 10 | 16 | 3 | 4 | 6 | 1692 | 1 |
| 11 | 22 | 5 | 16 | 7 | 217 | 3 |
| 12 | 141 | 5 | 29 | 4 | 121 | 1 |
| 13 | >2981 | 4 | 180 | 4 | 3275 | 1 |
| 14 | 12 | 4 | 8 | 4 | 8928 | 1 |
| 15 | 2201 | 4 | 1100 | 4 | 4309 | 1 |
| 16 | 74 | 3 | 69 | 3 | 2768 | 1 |
| 20 | 10983 | 4 | 2202 | 4 | | |
| 21 | 44 | 5 | 7 | 5 | | |
| 22 | 4046 | 4 | 957 | 4 | | |
| 23 | 680 | 5 | 109 | 4 | | |
| 24 | 41 | 3 | 28 | 3 | | |

¹“n” represents the number of times the compound was tested

### Assessment of the Contribution of DNL to Circulating and Sebum Lipids in Healthy Human Volunteers

The contribution of DNL to sebum and circulating lipids was assessed in a randomized, parallel study where 4 cohorts, each consisting of 5 subjects, were randomized into 4 arms differing in the timing of procedures. Subjects were male or female healthy volunteers between the ages of 18 and 50 years, inclusive. Healthy was defined as no clinically relevant abnormalities identified by a detailed medical history, full physical examination, including blood pressure (BP) and pulse rate measurement, 12-lead electrocardiogram (ECG), and clinical laboratory tests. Body mass index was 18 to 28 kg/m², inclusive.

Eligible subjects who met the entry criteria were admitted to the Clinical Trial Research Center (CTRC) on Day 0 for approximately 24 hours to receive multiple oral loading doses of deuterated water (²H₂O). During the inpatient stay at the CTRC, subjects received loading doses totaling 480 mL of 70% deuterated water divided into 8 doses of 60 mL each. These aliquots were administered orally every 3 hours over a 24-hour period. All subjects continued to take a daily oral deuterated water dose (60 mls of 70% ²H₂O) until Day 14. Subjects were asked to obtain a saliva sample on Day 2 for assessment of body water ²H₂O enrichment.

All subjects returned to the CTRC for 5 outpatient visits (including the follow-up visits). Subjects were randomized for sebum collections to occur on days 4 and 14, days 7 and 14, days 11 and 14, or on day 14. At all outpatient visits, blood samples were taken for determination of deuterated water enrichment in body water (from plasma) and for determination of VLDL-lipids. The staggered timing of blood and sebum collections allowed for assessment of the incorporation of deuterium into fatty acid contribution of DNL to lipid biosynthesis at different time points over a 14-day period. The goal was to establish a steady state to be used to assess the incorporation of deuterium into fatty acids and to assess the fractional contribution of DNL to lipid biosynthesis. Each cohort contributed data points to this continuum of DNL, whose time course had not yet been defined for sebaceous gland secretory products.

On sebum collection days, sebum lipids were collected using Sebutape®. Prior to application of the Sebutape®, the skin was cleansed of debris by washing with soap and water, and defatted by wiping with a gauze pad saturated in hexane. Once the skin was dry, the Sebutape® Test Strip was peeled from its backing paper using defatted forceps and affixed to the cleansed surface with gentle pressure to assure adequate adhesion. Surgical gloves were worn by the person handling the tape. Three patches were placed in the following areas: 1 patch on both cheeks (caudal of the middle line of the eye) and 1 on the forehead (cranial of the middle line of the eye). After 3 hours, the patches were removed and placed in acid-washed, Teflon-capped screw-cap vials.

Body water ²H₂O enrichment (precursor pool enrichment) was measured with the acetylene method in plasma and saliva samples (Previs et al., 1996). Plasma samples were also subjected to ultracentrifugation twice at 40,000 rpm for 30 min in a 50.4 Ti Beckman rotor at 10° C. to remove chylomicrons followed by a third ultracentrifugation step (40,000 rpm for 18 hours in a 50.4 Ti Beckman rotor at 10° C.) to isolate the very-low density lipoprotein (VLDL) fractions. The VLDL was then used for isolation of lipoprotein triglycerides (TG) by thin-layer chromatography (TLC). Total lipids were extracted with chloroform:methanol from sebum samples. VLDL-TG fatty acids and sebum total fatty acids were then trans-esterified to fatty acid methyl esters, in preparation for gas chromatographic/mass spectrometric (GC/MS) analysis. A DB-17 or equivalent column was used for isotope enrichment analysis of the fatty acid methyl esters with electron impact ionization ion at mass-to-charge ratio (m/z) 270-272, representing M0 through M2 isotopomers of palmitic acid. Excess M2 (EM2) and excess M1 (EM1) sample enrichments were determined by subtraction of natural abundance enrichment in unlabeled standards (run in parallel) from the sample enrichment.

The proportion of tissue palmitate derived from hepatic de novo lipogenesis (i.e., made “new” from acetyl-CoA precursors) was calculated by KineMed (Emeryville, Calif.) using mass isotopomer distribution analysis (MIDA). Briefly, EM1 was determined from the experimental data. Using this, the known n (number of repeating subunits in the polymer—22 for palmitate) and the measured p then allowed for the calculation of the asymptote (A*) or maximum possible palmitate enrichment if all the palmitate were newly synthesized using the known relationship between p and EM1. One then determines the fractional synthesis of palmitate by comparing the actual enrichment with the asymptote so:

Fractional palmitate DNL=EM1/A* or DNL (%)=EM1/A*×100

Data are presented as the percent contribution of novo synthesized palmitate over time to sebum lipids and to circulating lipids (VLDL triglycerides) with the values at or near steady state reflecting the percent contribution of DNL derived palmitate to the lipid pools. Data are shown in FIG. 1.

### Effect of Example 1 on Sebum Levels in Healthy Human Volunteers

Otherwise healthy overweight or obese subjects were admitted to the Clinical Research Institute and dosed orally with Example 1 (200 mg BID) or placebo for 14 consecutive days. On two days pre-treatment and days 13 and 14 (post-treatment) of the study, sebum production was assessed by Sebumeter® measurements and sebum was collected for lipid component identification and quantification. In order to prepare the area of the skin where the Sebumeter® measurements and the sebum collection will be performed, the skin surface has to be cleansed from any lipids and debris. The targeted area on the forehead and the cheeks were gently blotted using an oil-absorbing tissue or gauze. After blotting, wipes or gauze pads presoaked in 70% ethanol solution were used to wipe the forehead and cheeks. Following cleansing, the skin was allowed to dry and sebum measurements were made using a clean, calibrated Sebumeter® SM 815 (Courage+Khazaka, Köln, Germany) in accordance with the manufactures directions at 5 minutes and 3 hours post-cleansing. Sebum levels determined by Sebumeter® are expressed as relative change from pre-treatment baseline for subjects treated with placebo and Example 1 and are presented in FIG. 4.

The Sebutape® technique was used for sebum collection. At approximately 9 AM, following facial cleansing and the 5 minute post-cleanse sebumeter measurement, 4 Sebutape® strips were applied: 2 patches placed right and left on the forehead cranial of the middle line of the eye and 2 patches on the cheeks caudal of the middle line of the eye. The Sebutape® was peeled from its backing paper using defatted forceps and affixed to the surface with gentle pressure to assure adequate adhesion. Surgical gloves were to be worn by the person handling the tape. After 1 hour, the 4 patches were removed; the 2 left patches and the 2 right patches were placed in acid-washed, Teflon-capped screw-cap vials.

Sebum lipid species were quantified from the Sebutape® samples by Metabolon using their TrueMass® technology platform. Analysis of these sebum samples enabled assessment of the effect of Example 1 vs. placebo on individual sebum lipid classes. These data are expressed as relative change from pre-treatment baseline for subjects treated with placebo and Example 1. Individual subject level and box plots depicting means with confidence intervals are shown for sebum triglycerides, wax esters, and free fatty acids (FIG. 5).

### Assessment of the Impact of ACCi on Malonyl-CoA Levels in Syrian Hamsters

Male Syrian gold Hamster at ˜10 weeks were maintained adlibitum on standard hamster chow and water. Example 3 for topical delivery was prepared as a solution in a vehicle consisting of 70:30 (Ethanol:propylene glycol) at a concentration of 100 mg/ml. Topical administration of a single dose (5 ul/ear) was applied to hamster ears with a pipette (2-10 ul) tip adhering to 2 minute intervals following the dosing order. The dosing timing allowed for the formulation to spread evenly over an approximate 1 sq cm area and absorb/dry prior to returning the animal to the cage. Similarly for oral dosing, at 2 hours into the light cycle a dose volume of 10 mL/kg was administered to deliver 100 mg/kg Example 8 or vehicle (1% hydroxypropyl methylcellulose acetate succinate in 20 mM Tris pH 7.4) as a single oral gavage to each hamster in 2 minute intervals. One hour after dose administration animals were sacrificed by CO₂ asphyxiation.

Briefly, ear skin samples were prepared for malonyl-CoA analysis in the following manner. One 8 mm distal biopsy punch (using a 8 mm diameter Sklar Tru-punch—Sklar Instruments) (2 per animal) was taken just above the anatomical “V” in the aural cartilage to normalize sample area. The punch was then split into 2 layers (anterior and posterior). The anterior (front) surface was rapidly frozen (−80 C) and retained for analysis.

Frozen tissues were homogenized in 1 mL of 5% ice cold trichloroacetic acid using a polytron in a 2 mL polypropylene centrifuge tube. A 10 μL aliquot of intermediate internal standard solution was spiked into each homogenate and final calibration solution and vortexed briefly. Homogenates were centrifuged at 14,000 rpm at 4° C. for 5 min. Prior to solid phase extraction, Waters C18 Oasis (30 mg) solid phase extraction cartridges were conditioned with 1 mL of methanol followed by 1 mL of water using a Waters glass vacuum manifold. The supernatants and calibrator solutions were loaded onto the solid phase extraction cartridges followed by washing with 2.5 mL of water and elution with 1 mL of methanol into 13×100 mm glass test tubes. The methanol supernatants were loaded onto a 1 mL, 96-well polypropylene plate and evaporated under nitrogen at 30° C. Samples were reconstituted with 100 μL of 10 mM ammonium bicarbonate (pH 9.5) and vortexed for 2 minutes in a plate vortexer after sealing the 96-well plate. Tissue malonyl-CoA levels were determined by LCMS by the Metabolomics Laboratory Sanford-Burnham Medical Research Institute (Orlando, A). Hamster ear skin malonyl-CoA levels were expressed as the percent of the vehicle control and are plotted in FIG. 6.

### Assessment of the Contribution of DNL to Circulating and Sebum Lipids in Syrian Hamsters

Male Syrian gold hamsters weighing between 150-200 g (22-23 weeks) were maintained adlibitum on standard hamster chow and on a 12 hour light and dark cycle. At the start of ²H₂O labeling animals were injected intraperitoneal with an 8% ²H₂O solution at 3.5 ml/kg in the morning and maintained thereafter with 8% ²H₂O adlibitum for either 1, 4, 7, 14 or 20 days (n=6 each day). After completion of the appropriate labeling period, hamsters were sacrificed by CO₂ asphyxiation and tissues were removed and snap-frozen in liquid nitrogen. Approximately 4-5 ml of blood was obtained at sack via cardiac stick and then transferred to a BD Vacutainer Plus Plastic K₂EDTA tube (cat #368589). Plasma was separated from blood by centrifugation at 1300 RCF for 10 minutes at room temperature and immediately transferred to a separate tube and frozen on dry ice. Hamster pinia (ear) were removed by punch biopsy (using an 8 mm diameter Sklar Tru-punch—Sklar Instruments). Punch biopsies were taken just above the anatomical “V” in the aural cartilage of each ear to standardize sample collection area. The punch was then split into 2 layers (anterior and posterior). The anterior (front) surface was retained for analysis. For liver samples the bifurcated medial lobe was removed, rinsed in saline and drained of blood by placing the cut end of the lobe on a sterile absorbent paper and using capillary action. The liver lobe was then flash frozen in liquid nitrogen. Tissues and plasma were sent to KineMed (Emeryville, Calif.) for determination of the percent contribution of DNL to the sebum and circulating lipids using mass isotopomer distribution analysis (MIDA) (Hellerstein, 1999). Data are presented as the percent contribution of novo synthesized palmitate over time to sebum lipids and to circulating lipids (plasma triglycerides) with the values at or near steady state reflecting the percent contribution of DNL derived palmitate to the lipid pools. Data are shown in FIG. 7.

### Effect of Oral and Topical ACCi on DNL in Syrian Hamsters

Male Syrian gold Hamster at ˜10 weeks were maintained adlibitum on standard hamster chow and water. On the day of the experiment at 2 hours into the light cycle a dose volume of 10 mL/kg was administered to deliver 100 mg/kg Example 8 or paired vehicle (1% hydroxypropyl methylcellulose acetate succinate in 20 mM Tris pH 7.4) as a single oral gavage to each hamster in 2 minute intervals. Example 3 for topical delivery was prepared as a solution in a vehicle consisting of 70:30 (Ethanol:propylene glycol) at a concentration of 100 mg/ml. Topical administration of a single dose (5 ul/ear) was applied to hamster ears with a pipette (2-10 ul) tip adhering to 2 minute intervals following the dosing order. The dosing timing allowed for the formulation to spread evenly over an approximate 1 sq cm area and absorb/dry prior to returning the animal to the cage. 1 hour post ACCi dose each animal received an IP injection of ¹⁴C-labeled acetate (ARC0158B diluted in saline) following the 2 minute interval timing format. Each animal received an individually calculated amount of ¹⁴C-acetate based on body weight (0.1 μCi/g in a dosing volume of 2 μl/g). One hour after the ¹⁴C-Acetate injection animals were sacrificed by CO₂ asphyxiation.

Liver and ear skin were collected for de novo lipogenesis determinations (¹⁴C incorporation to lipid). Briefly, 2 liver punches totaling ˜400 mg of liver were collected from the bifurcated median lobe of each animal (using a 8 mm diameter Sklar Tru-punch—Sklar Instruments), rinsed with saline and blotted dry. The tissues were placed into pre-weighed in glass tubes (Pyrex 9826-16×125 mm with PTFE lined caps) containing NaOH (1.5 mL of 2.5 M).

Ear skin samples were prepared for analysis in the following manner. One 8 mm distal biopsy punch (using an 8 mm diameter Sklar Tru-punch—Sklar Instruments) (2 per animal) was taken just above the anatomical “V” in the aural cartilage to standardize sample collection area. The punch was then split into 2 layers (anterior and posterior). The anterior (front) surface is retained for analysis. The anterior skin was placed into pre-weighed in glass tubes (Pyrex 9826-16×125 mm with PTFE lined caps) containing NaOH (1.5 mL of 2.5 M).

Upon completion of the study, the liver and ear skin samples in NaOH were weighed and this weight was used to calculate the mass of the tissue collected. The capped tubes were heated in an dry oven (˜60° C.) until the tissue was fully degraded (˜4-6 hr, gentle vortexing 2-3 times during heating). Following degradation and cooling absolute ethanol (2.5 mL) was added to each sample. The tubes were recapped and vigorously mixed (vortexed) for 60 seconds and allowed to settle overnight at RT. Petroleum ether (4.8 mL) was added to each tube, recapped and the samples were vigorous mixed (60 sec). The samples were centrifuged in the Sorvall RT6000 (1500×g for 5 min) to separate the organic and aqueous phases. The resulting upper organic phase was removed through gentle aspiration and discarded. Concentrated HCl (0.6 mL of 12M) was added to the remaining aqueous phase of each sample (including the interface material), capped and vortexed vigorously for 60 sec. The acidified aqueous phase was extracted with petroleum ether (4.8 mL) and then centrifuged in the Sorvall RT6000 (1500×g for 5 min) to separate the organic and aqueous phases. The upper organic phase was removed/collected in a 20 mL scintillation vial and capped. The remaining aqueous phase (including the interface material) was again extracted with petroleum ether (4.8 mL). Following 60 sec. of vigorous vortexing the samples were centrifuged in the Sorvall RT6000 (1500×g for 5 min) to separate the organic and aqueous phases. The upper organic phase was removed and pooled with the previous extraction in the 20 ml scintillation vial. The pooled organic extractions were evaporated to dryness under gentle flow of N₂ (˜2 hr) at RT. Aquasol-2 scintillation fluid (10 mL) (or other compatible scintillation fluid) was added to each vial and after vortexing the samples were counted in an appropriate scintillation counter (e g. Wallac Rack-Beta 1409 LSC).

The conversion of ¹⁴C-actetate to ¹⁴C counts present in the organic phase post extraction represented DNL. Data were expressed as the percent of the vehicle control and are plotted in FIG. 8.

### Assessing the Effects of ACC Inhibition on Hamster Ear Triglyceride Content in Syrian Hamsters

Male Syrian gold hamsters weighing between 150-200 g (22-23 weeks) were maintained adlibitum on standard hamster chow and on a 12 hour light and dark cycle. Hamsters were treated with either 30 mg/kg of Example 8 or vehicle (1% hydroxypropyl methylcellulose acetate succinate in 20 mM Tris pH 7.4) once daily for 19 days. At the end of the study period, animals were euthanized by CO₂ asphyxiation. Hamster pinia (ear) were removed by punch biopsy (using an 8 mm diameter Sklar Tru-punch—Sklar Instruments). Punch biopsies were taken just above the anatomical “V” in the aural cartilage of each ear to standardize sample collection area. The punch was then split into 2 layers (anterior and posterior). The anterior (front) surface was retained for analysis. For liver samples the bifurcated medial lobe was removed, rinsed in saline and drained of blood by placing the cut end of the lobe on a sterile absorbent paper and using capillary action. The liver lobe was then flash frozen in liquid nitrogen. Two Qiagen 3 mm Tungsten beads were added to tubes containing individual anterior biopsies of hamster ear skin along with 500 ul of homogenization buffer (methanol:water 1:1 v/v). Samples were homogenized for 5 minutes at a frequency of 25 on the Qiagen Tissuelyser. Ear skin triglyceride content was determined via LC-MS on the AB SCIEX Qtrap 5500. Following homogenization, lipids were extracted from the homogenate with Dichloromethane:lsopropanol:Methanol (25:10:65, v/v/v) containing the following internal standards at a concentration of 200 nM: Glyceryl Triheptadecanoate, 1,2-Dinonadecanoin, Cholesteryl Heptadecanoate, 1,2-Dilauroyl-sn-glycero-3-phosphocholine, 1-Heptadecanoyl-2-hydroxy-sn-glycero-3-phosphocholine, and Palmitoyl-L-carnitine-(N-methyl-d₃) hydrochloride. Lipid extracts were then analyzed by UPLC-MS/MS using a Waters Acquity UPLC coupled to an AB Sciex QTRAP 5500 mass spectrometer. Lipid classes were separated by reversed-phase chromatography on a Waters Acquity UPLC BEH300 C4 column, 1.7 um, 2.1×50 mm. Lipid species were then analyzed on the mass spectrometer using positive ion electrospray ionization in the multiple reaction monitoring (MRM) mode. LC chromatogram peak integration was performed with AB Sciex MultiQuant software. Hamster ear skin triglyceride levels (mg/g tissue) were plotted for vehicle and Example 8 in FIG. 9.

### Pharmacokinetic Drug Interactions

The ability of Example 1 to inhibit seven major P450 isoforms (CYP3A, CYP1A2, CYP2B6, CYP2C8, CYP2C9, CYP2C19, and CYP2D6) was investigated using patient liver microsomes and probe substrates. Based on IC₅₀ values of >30 μM determined from in vitro studies, Example 1 is not predicted to demonstrate competitive pharmacokinetic drug interactions with compounds for which CYP3A, CYP1A2, CYP2B6, CYP2C8, CYP2C9, CYP2C19, or CYP2D6 constitute the primary mechanism of clearance.

### Human Studies Discussion

Example 1 was administered to 180 subjects and found to be safe and generally well tolerated. Single oral doses up to 800 mg (divided dose) were administered to healthy lean and overweight subjects, and repeated doses up to 400 mg (administered as 200 mg BID) for up to 14 days were administered to healthy and type 2 diabetic overweight and obese subjects. There were no dose- or duration-related increase in the frequency of adverse events observed, and the maximum tolerated dose was not established. The oral absorption of Example 1 was rapid with median Tmax occurring at approximately 1-2 hours post dose in the fasted state and approximately 3-4 hours in the fed state. Food modestly decreased the rate but not the extent of Example 1 absorption supporting dosing without regard to timing of food. Terminal phase half-life for Example 1 was approximately 10-13 hours. Exposures (AUC and Cmax) increased dose-proportionally with single doses up to 600 mg.

A methodology study was conducted in healthy subjects to evaluate the relative contribution of DNL to various lipid pools including very-low-density-lipoprotein-triglyceride (VLDL-TG) and sebum. The study revealed that patient sebum, relative to other lipid pools, was highly dependent on localized DNL.

Example 1 lowered sebum levels >49% from baseline in treated subjects relative to placebo. Further analysis of specific lipid classes demonstrated that sebum triglycerides, the major lipid class in sebum, were decreased by 66% relative to placebo. Levels of sebum free fatty acids and wax esters, which are also dependent on DNL, were reduced in Example 1 treated subjects relative to placebo treated subjects by approximately 49% and 53% respectively.

In summary, Example 1 is a dual ACC1/ACC2 inhibitor that dose-dependently suppressed DNL in healthy human volunteers by up to 80% reducing production of sebum by 49% compared to baseline (FIG. 4). Analysis of specific lipid classes demonstrated that sebum triglycerides, the major lipid class in sebum, were decreased by 66% (FIG. 5). Levels of sebum free fatty acids and wax esters, which are also dependent on DNL, were also reduced in Example 1 treated subjects relative to placebo treated subjects (FIG. 5). In contrast, free cholesterol, which is not dependent on DNL, showed no change relative to placebo. Squalene levels, which are also not dependent on DNL, showed a 2.6-fold increase relative to placebo.

### Radiometric ACC1 and ACC2 Inhibition Assay Description

For ACC inhibition studies, test compounds were dissolved in dimethyl sulfoxide (DMSO) and serially diluted in DMSO in order to run in a 11-point dose response with final compound concentration ranging from 10 μM to 0.3 nM. Aliquots of 1 μL were added in replicate to 96 well plates and an equal volume of DMSO was added to control wells. The enzyme solution was activated for 30 minutes at 37° C. in buffer containing 50 mM HEPES (pH 7.5), 10 mM MgCl₂, 10 mM tripotassium citrate, 6 mM DTT, 0.75 mg/mL BSA, and 0.8 μg/mL ACC. After a ten minute enzyme-compound preincubation, the reaction was initiated at room temperature in a fume hood by addition of the substrate solution (containing 2.4 mM acetyl-CoA, 38.4 mM KHCO₃, 1.6 mM NaH[¹⁴C]O₃, and 8.0 mM ATP). The final assay volume of 100 μL per well consisted of: 46 mM HEPES (pH 7.5), 7.5 mM MgCl₂, 7.5 mM tripotassium citrate, 2.8 mM DTT, 0.5 mg/mL BSA, 2.0 mM ATP, 600 μM acetyl-CoA trilithium salt, 9.6 mM potassium bicarbonate, 0.6 μg/mL hACC1 or 2, and 0.4 mM NaH[¹⁴C]O₃ (58 mCi/mmol). After 20 minutes, the reaction was terminated by the addition of 3 N hydrochloric acid (HCl) with the concomitant liberation of non-reacted NaHCO₃ as CO₂. Plates were dried overnight at 50° C. to allow complete [¹⁴C]O₂ liberation. The following day, 30 μL of water was added to the dried wells now containing [¹⁴C]malonyl-CoA, followed by 95 μL of Opti Phase Supermix liquid scintillation fluid. The plates were shaken vigorously, sealed, and transferred to a Microbeta LSC luminescence counter to quantify the amount of ¹⁴C in each assay well.

Results of the radiometric assay are shown in Table 2.

TABLE 2

| | Radiometric | | Radiometric | |
|---|---|---|---|---|
| | Assay | | Assay | |
| Example | hACC1 IC₅₀ | | hACC2 IC₅₀ | |
| Number | (nM) | n¹ | (nM) | n¹ |
| 1 | 11 | 5 | 27 | 6 |
| 2 | 3 | 4 | 4 | 4 |
| 3 | 5 | 3 | 5 | 3 |
| 4 | 6 | 3 | 5 | 3 |
| 5 | 5 | 4 | 8 | 4 |
| 6 | 5 | 3 | 4 | 3 |
| 7 | 11 | 3 | 4 | 4 |
| 8 | 19 | 3 | 10 | 3 |
| 9 | 140 | 4 | 16 | 4 |
| 11 | 4 | 3 | 7 | 3 |
| 12 | 26 | 3 | 2 | 3 |
| 14 | 23 | 3 | 5 | 3 |
| 17 | >10,000 | 1 | | |
| 18 | >10,000 | 1 | | |
| 19 | >10,000 | 1 | | |
| 21 | 98 | 6 | 2 | 6 |
| 23 | 687 | 2 | 16 | 2 |

¹“n” represents the number of times the compound was tested

1. Corbett J W. Review of recent acetyl-CoA carboxylase inhibitor patents: mid-2007-2008. Expert Opin Ther Pat. 2009; 19:943-56.
2. Dawson A L, Dellavalle R P. Acne vulgaris. BMJ. 2013; 346:f2634.
3. Downie M M, Kealey T. Lipogenesis in the human sebaceous gland: glycogen and glycerophosphate are substrates for the synthesis of sebum lipids. J Invest Dermatol. 1998; 111:199-205.
4. Freeman-Cook K D, Amor P, Bader S, Buzon L M, Coffey S B, Corbett J W, Dirico K J, Doran S D, Elliott R L, Esler W, Guzman-Perez A, Henegar K E, Houser J A, Jones C S, Limberakis C, Loomis K, McPherson K, Murdande S, Nelson K L, Phillion D, Pierce B S, Song W, Sugarman E, Tapley S, Tu M, Zhao Z. Maximizing lipophilic efficiency: the use of Free-Wilson analysis in the design of inhibitors of acetyl-CoA carboxylase. J Med Chem. 2012; 55:935-42
5. Gannon M, Underhill M, Wellik K E. J. Family Pract. 2011; 60:290-92.
6. Geiger J M. Retinoids and sebaceous gland activity. Dermatology. 1995; 191:305-10.
7. Glund S, Schoelch C, Thomas L, Niessen H G, Stiller D, Roth G J, Neubauer H. Inhibition of acetyl-CoA carboxylase 2 enhances skeletal muscle fatty acid oxidation and improves whole-body glucose homeostasis in db/db mice. Diabetologia. 2012; 55:2044-53
8. Harwood H J Jr. Treating the metabolic syndrome: acetyl-CoA carboxylase inhibition. Expert Opin Ther Targets. 2005; 9:267-81.
9. Harwood H J Jr, Petras S F, Shelly L D, Zaccaro L M, Perry D A, Makowski M R, Hargrove D M, Martin K A, Tracey W R, Chapman J G, Magee W P, Dalvie D K, Soliman V F, Martin W H, Mularski C J, Eisenbeis S A. Isozyme-nonselective N-substituted bipiperidylcarboxamide acetyl-CoA carboxylase inhibitors reduce tissue malonyl-CoA concentrations, inhibit fatty acid synthesis, and increase fatty acid oxidation in cultured cells and in experimental animals. J Biol Chem. 2003; 278:37099-111.
10. Hellerstein M K, Neese R A. Mass isotopomer distribution analysis at eight years: theoretical, analytic, and experimental considerations. Am J Physiol. 1999; 276:E1146-70.
11. Hellerstein M K. De novo lipogenesis in humans: metabolic and regulatory aspects. Eur J Clin Nutr. 1999; 53 Suppl 1:S53-65.
12. Janiczek-Dolphin N, Cook J, Thiboutot J, Clucas A. Can Sebum reduction predict acne outcome? Br J Dermatol. 2010; 163:683-688.
13. Jones P J. Tracing lipogenesis in humans using deuterated water. Can J Physiol Pharmacol. 1996; 75: 755-60.
14. Kligman A M, Miller D L, McGinley K J. Sebutape®: A device for visualizing and measuring human sebaceous secretion. J Soc Cosmet Chem. 1986; 37:369-74.
15. Lee W N, Bassilian S, Ajie H O, Schoeller D A, Edmond J, Bergner E A, Byerley L O. In vivo measurement of fatty acids and cholesterol synthesis using D2O and mass isotopomer analysis. Am J Physiol 1994; 266: E600-E708.
16. Ottaviani M, Camera E, Picardo M. Lipid mediators in acne. Mediators of Inflammation. 2010. doi:10.1155/2010/858176 17. Plewig G, Luderschmidt C. Hamster ear model for sebaceous glands. J Invest Dermatol. 1977; 68:171-6.
18. Zouboulis C C, Seltmann H, Neitzel H, Orfanos C E. Establishment and characterization of an immortalized human sebaceous gland cell line (SZ95). J Invest Dermatol. 1999; 113:1011-20.
19. Zouboulis C C. Acne and sebaceous gland function. Clin. Dermatol. 2004; 22:360-66.

We claim:

1. Use of an ACC inhibitor, or a pharmaceutically acceptable salt thereof, in the manufacture of a medicament for the treatment of acne.

2. Use of a pharmaceutical composition comprising an ACC inhibitor, or a pharmaceutically acceptable salt thereof, and at least one pharmaceutically acceptable carrier thereof in the manufacture of a medicament for the treatment of acne.

3. Use of an ACC inhibitor, or a pharmaceutically acceptable salt thereof, in the manufacture of a medicament for reducing sebum triglycerides, sebum free fatty acids, cholesterol esters and sebum waxy esters in a patient.

4. Use of a pharmaceutical composition comprising an ACC inhibitor, or a pharmaceutically acceptable salt thereof, and at least one pharmaceutically acceptable carrier thereof in the manufacture of a medicament for reducing sebum triglycerides, sebum free fatty acids, cholesterol esters and sebum waxy esters in a patient.

5. Use of 1′-(2-aminoquinoline-7-carbonyl)-1-isopropyl-4,6-dihydrospiro[indazole-5,4′-piperidin]-7(1H)-one, or a pharmaceutically acceptable salt thereof, in the manufacture of a medicament for the treatment of acne.

6. Use of a pharmaceutical composition comprising 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier thereof in the manufacture of a medicament for the treatment of acne.

7. Use of 1′-(2-aminoquinoline-7-carbonyl)-1-isopropyl-4,6-dihydrospiro[indazole-5,4′-piperidin]-7(1H)-one, or a pharmaceutically acceptable salt thereof, in the manufacture of a medicament for reducing sebum triglycerides, sebum free fatty acids, cholesterol esters and sebum waxy esters in a patient.

8. Use of a pharmaceutical composition comprising 1′-(2-aminoquinoline-7-carbonyl)-1-isopropyl-4,6-dihydrospiro[indazole-5,4′-piperidin]-7(1H)-one, or a pharmaceutically acceptable salt thereof, and at least one pharmaceutically acceptable carrier thereof in the manufacture of a medicament for reducing sebum triglycerides, sebum free fatty acids, cholesterol esters and sebum waxy esters in a patient.


SEQUENCE LISTING (2 sequences)

SEQ ID NO: 1 - protein, 2356 aa, Homo sapiens
MAHHHHHHDEVDDEPSPLAQPLELNQHSRFIIGSVSEDNSEDEISNLVKLDLLEKEGSLSPASVGSDTLSDLGISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGVVCGALHVADVSLRNSVSNFLHSLERGQVLPAHTLLNTVDVELIYEGVKYVLKVTRQSPNSYVVIMNGSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQLDNPSKVQQAELHTGSLPRIQSTALRGEKLHRVFHYVLDNLVNVMNGYCLPDPFFSSKVKDWVERLMKTLRDPSLPLLELQDIMTSVSGRIPPNVEKSIKKEMAQYASNITSVLCQFPSQQIANILDSHAATLNRKSEREVFFMNTQSIVQLVQRYRSGIRGHMKAVVMDLLRQYLRVETQFQNGHYDKCVFALREENKSDMNTVLNYIFSHAQVTKKNLLVTMLIDQLCGRDPTLTDELLNILTELTQLSKTTNAKVALRARQVLIASHLPSYELRHNQVESIFLSAIDMYGHQFCIENLQKLILSETSIFDVLPNFFYHSNQVVRMAALEVYVRRAYIAYELNSVQHRQLKDNTCVVEFQFMLPTSHPNRGNIPTLNRMSFSSNLNHYGMTHVASVSDVLLDNSFTPPCQRMGGMVSFRTFEDFVRIFDEVMGCFSDSPPQSPTFPEAGHTSLYDEDKVPRDEPIHILNVAIKTDCDIEDDRLAAMFREFTQQNKATLVDHGIRRLTFLVAQKDFRKQVNYEVDRRFHREFPKFFTFRARDKFEEDRIYRHLEPALAFQLELNRMRNFDLTAIPCANHKMHLYLGAAKVEVGTEVTDYRFFVRAIIRHSDLVTKEASFEYLQNEGERLLLEAMDELEVAFNNTNVRTDCNHIFLNFVPTVIMDPSKIEESVRSMVMRYGSRLWKLRVLQAELKINIRLTPTGKAIPIRLFLTNESGYYLDISLYKEVTDSRTAQIMFQAYGDKQGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 2 - protein, 2458 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST